(19) United States
(12) Patent Application Publication
(10) Pub. No.: US 2003/0220266 A1
(43) Pub. Date: Nov. 27, 2003

(54) Method of treating a disease

(76) Inventors: Kurt Jason Sales, Edinburgh (GB); Henry Nicolas Jabbour, Edinburgh (GB); Arieh Katz, Cape Town (ZA)

Correspondence Address: Michael L. Goldman, NIXON PEABODY LLP, Clinton Square, P.O. Box 31051, Rochester, NY, 14603-1051, US

(21) Appl. No.: 10/284,569

(22) Filed: Oct. 30, 2002

Related U.S. Application Data
(60) Provisional application No. 60/340,971, filed on Oct. 30, 2001.

(51) Int. Cl. A61K 38/08; A61K 31/405; A61K 31/192

(52) U.S. Cl. 514/15; 514/420; 514/569; 514/570; 514/16

(57) ABSTRACT

A method of treating a neoplastic condition of the cervix in a patient the method comprising administering to the patient an inhibitor of cyclooxygenase-1 (COX-1) and/or an EP2 and/or EP4 receptor antagonist

The present invention relates to a method of treating a disease, and in particular to a method of treating a neoplastic condition of the cervix.

Cancer of the uterine cervix is one of the leading causes of cancer-related death in women world-wide. Cervical cancer is particularly common in less developed countries, including South and Central America, Southeast Asia and Sub-Saharan Africa. Histopathologic classification of cervical cancer precursors is generally using the CIN nomenclature (cervical intraepithelial neoplasia). The nomenclature CIN grades I to III are used to describe pre-invasive epithelial lesions or various categories of dysplasia and carcinoma in situ. CIN grade I is equivalent to mild dysplasia in which undifferentiated cells occupy approximately the lower one third of the epithelium. CIN grade II is equivalent to moderate dysplasia where undifferentiated cells replace two thirds of the thickness of normal epithelium. CIN grade III denotes severe dysplasia and carcinoma in situ.

Symptoms of cervical carcinoma include vaginal bleeding, persistent so sanguinous foul-smelling discharge, disruption of normal bladder or bowel function and leg oedema. The WHO recognises three general categories of cervical cancer, namely squamous cell carcinoma, adenocarcinoma and other epithelial tumours including less common types such as adenosquamous carcinoma, glassy cell carcinoma, adenoid basal cell carcinoma as well as carcinoid-like and small cell carcinoma. Adenocarcinomas and to a lesser extent adenosquamous carcinomas and rarely sarcomas, lymphoma and melanoma account for approximately 20% of the invasive cervical carcinoma and display a variety of types and sub-types.

Cyclooxygenases (sometimes called prostaglandin endoperoxide synthase) are involved in prostaglandin synthesis. Two cyclooxygenase isoenzymes, COX-1 and COX-2, have been identified. COX-1 expression is considered to be constitutive, as basal levels of COX-1 mRNA and protein are observed to be present and generate prostaglandins for normal physiological functions. In contrast, COX-2 expression is inducible.

Cyclooxygenases have been studied in various cancers, but the picture that has emerged to date is confusing and it is not possible to predict the role of COX-1 or COX-2 in any particular cancer. For example, both COX-1 and COX-2 have been shown to be highly expressed in lung cancer in the mouse (Bauer et al (2000) Carcinogenesis 21, 543-550) whereas Rioux & Castonguay (2000) Carcinogenesis 21, 1745-1751 indicates that COX-1 is induced by tobacco carcinogens in human macrophages and is correlated with NFκB activation. According to Doré et al (1998) J. Histochem. Cytochem. 46, 77-84 COX-1 but not COX-2 is expressed in human ovarian adenocarcinomas. According to Ryu et al (2000) Gynecologic Oncology 76, 320-325 COX-2 expression is high in stage 1D cervical cancer whereas COX-1 was expressed without regard to location of the tumour cells or type of cancer cell and the authors indicate that COX-1 is unrelated to apoptosis, tumourigenesis and tumour invasion mechanisms. Kulkarni et al (2001) Clin. Cancer Res. 7, 429-434 indicates that COX-2 is overexpressed in human cervical cancer,

Prostaglandin E2 elicits its autocrine/paracrine effects on target cells through interaction with transmembrane G protein coupled receptors. To date four main sub-types of PGE₂ receptors have been identified based on responses to agonists and antagonists and are pharmacologically divided into EP1, EP2, EP3 and EP4 which utilise alternate and in some cases opposing intracellular signalling pathways. EP2 and EP4 increase cAMP levels via G_{αs}.

The inventors have surprisingly found that COX-1 is upregulated in cervical carcinoma as are the EP2 and EP4 receptors, and basal cAMP levels are signficantly higher in cervical carcinoma tissues compared to normal cervix. The inventors propose new methods of treating a neoplastic condition of the cervix, by which we include CIN grades I to II, carcinoma in situ and invasive carcinoma.

A first aspect of the invention provides a method of treating a neoplastic condition of the cervix in a patient the method comprising administering to the patient an inhibitor of cyclooxygenase-1 (COX-1).

A second aspect of the invention provides a method of treating a neoplastic condition of the cervix in a patient the method comprising administering to the patient an EP2 or EP4 receptor antagonist.

By “a neoplastic condition of the cervix” we specifically include CIN grades I to III, carcinoma in situ and invasive carcinoma. Thus, the carcinoma may be at any stage of differentiation. We also specifically include the three general categories of cervical cancer, namely squamous cell carcinoma, adenocarcinoma and other epithelial tumours including less common types such as adenosquamous carcinoma, glassy cell carcinoma, adenoid basal cell carcinoma as well as carcinoid-like and small cell carcinoma. The cervix is the uterine cervix.

It is particularly preferred if the method of the invention is used to treat adenocarcinoma or squamous cell carcinoma of the cervix.

The patient may be any patient who is suffering from a neoplastic condition of the cervix or a patient who is at risk from this. Risk factors include human papilloma virus infection (although 20% of women developing cervical carcinoma do not have HPV infection), smoking, having more than one sexual partner and a family history of the disease. The patient to be treated may be any female individual who would benefit from such treatment. Typically and preferably the patient to be treated is a human female. However, the methods of the invention may be used to treat female mammals, such as the females of the following species: cows, horses, pigs, sheep, cats and dogs. Thus, the methods have uses in both human and veterinary medicine.

When the treatment is used prohylactically (as discussed in more detail below), typically women at risk are treated, for example, women with HPV infection and/or who have a family history of cervical cancer.

We have found up-regulation of expression of cyclooxygenase-2 and prostaglandin E receptors (EP2 and EP4) in cervical carcinoma cells by seminal plasma which containd hich levels of PGE₂. Thus, the methods of the invention using an EP2 and/or EP4 receptor antagonist is particularly suited for use in sexually active women, particularly those who do not use a barrier method of contraception. However, the findings of the inventors suggest that it may be additionally helpful in the treatment if the woman abstains or uses a barrier method of contraception.

The inhibitor of COX-1 may be any suitable inhibitor of COX-1. By “suitable” we mean that the inhibitor may be administered to the patient. It will be appreciated that one or more inhibitors of COX-1 may be administered to the patient. Non-steroidal anti-inflammatory drugs (NSAIDs) typically are inhibitors of COX-1. Many NSAIDs are known in the art, and include those disclosed in Martindale's, The complete drug reference, 32^{nd} Edition, Parfitt, K (ed), Pharmaceutical Press, London, UK, incorporated herein by reference. NSAIDs (COX-1 inhibitors) include ibuprofen, naproxen, fenbufen, fenoprofen, flurbiprofen, ketoprofen, dexketoprofen, tiaprofenic acid, azapropazone, diclofenac, aceclofenac, diflunisal, etodolac, indomethacin, ketorolac, mefenamic acid, meloxicam, namubetone, phenylbutazone, piroxicam, sulindac, tenoxicam and tolfenamic acid.

It is preferred if the inhibitor is selective for COX-1 over COX-2. By “selective” we mean that the inhibitor inhibits COX-1 at least ten times more effectively than COX-2. Mofezolac ([3,4-di(4-methoxyphenyl)-5-isoxazloyl]acetic acid; available from Mitsubishi Pharma, Chikujo-gun, Fukuoku, Japan) is a COX-1 selective inhibitor. The ratio of the mofezolac IC₅₀ values for ovine COX-1 to COX-2 is reported to be 0.003:1 (Goto et al (1998) Prostaglandins Other Lipid Mediat. 56, 245-254).

SC560 is a selective COX-1 inhibitor available from Pharmacia (St Louis, Mo., USA).

The prostaglandin EP2 receptor antagonist may be any suitable EP2 receptor antagonist. Similarly, the prostaglandin EP4 receptor antagonist may be any suitable EP4 receptor antagonist. By “suitable” we mean that the antagonist is one which may be administered to the patient. The receptor antagonists are molecules which bind to their respective receptors, compete with the natural ligand (PGE₂) and inhibit the initiation of the specific receptor-mediated signal transduction pathways. The receptor antagonists are typically selective to the particular receptor and typically have a higher binding affinity to the receptor than the natural ligand. Although antagonists with a higher affinity for the receptor than the natural ligand are preferred, antagonists with a lower affinity may also be used, but it may be necessary to use these at higher concentrations. Preferably, the antagonists bind reversibly to their cognate receptor. Typically, antagonists are selective for a particular receptor and do not affect the other receptor; thus, typically, an EP2 receptor antagonist binds the EP2 receptor but does not substantially bind the EP4 receptor, whereas an EP4 receptor antagonist binds the EP4 receptor but does not substantially bind the EP2 receptor. Preferably, the EP2 or fEP4 receptor antagonist is selective for the particular receptor subtype. By this is meant that the antagonist has a binding affinity for the particular receptor subtype which is at least ten-fold higher than for at least one of the other EP receptor subtypes. Thus, selective EP4 receptor antagonists have at least a ten-fold higher affinity for the EP4 receptor than any of the EP1, EP2 or EP4 receptor subtypes.

EP2 receptor antagonists include AH6809 (Pelletier et at (2001) Br. J. Pharmacol. 132, 999-1008).

EP4 receptor antagonists include AH23848B (developed by Glaxo) and AH22921X (Pelletier et al (2001) Br. J. Pharmacol. 132, 999-1008. The chemical name for AH23848B3 is ([1alpha(z), 2beta5alpha]-(+/−)-7-[5-[[(1,1′-biphenyl)-4-yl]methoxy]-2-(4-morph olinyl)-3-oxo-cyclopentyl]-4-heptenoic acid) (see Hillock & Crankshaw (1999) Eur. J. Pharmacol. 28, 99-108). EP4RA (Li i (2000) Endocrinology 141, 205461) is an EP(4)-selective ligand (Machwate et al (2001) Mol. Pharmacol. 60:36-41). The omega-substituted prostaglandin E derivatives described in WO 00/15608 (EP 1 114 816) (Ono Pharm Co Ltd) bind EP4 receptors selectively and may be EP4 receptor antagonists.

Peptides described in WO 01/42281 (Hopital Sainte-Justine) eg: IFTSYLECL (SEQ ID No 1), IFASYECL (SEQ ID No 2), IFTSAECL (SEQ ID No 3), IFTSYEAL (SEQ ID No 4), ILASYECL (SEQ ID No 5), IFTSTDCL (SEQ ID No 6), TSYEAL (SEQ ID No 7) (with 4-biphenyl alanine), TSYEAL (with homophenyl alanine) are also described as EP4 receptor antagonists, as are some of the compounds described in WO 00/18744 (Fujisawa Pharm Co Ltd). The 5-thia-prostaglandin E derivatives described in WO 00/03980 (EP 1 097 922) (Ono Pharm Co Ltd) may be EP4 receptor antagonists.

EP4 receptor antagonists are also described in WO 01/10426 (Glaxo), WO 00/21532 (Merck) and GB 2 330 307 (Glaxo).

WO 00/21532 describes the following as EP4 receptor antagonists:

5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt;

5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one;

5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one;

5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one;

5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

GB 2 330 307 describes [1α(Z), 2β,5α]-(±)-7-1[5-[[(1,1′-biphenyl)-4-yl]methoxy]-2-(4-morpholinyl)-3-oxocyclopentyl]-4-heptenoic acid and [1R[1α(z),2β,5α]]-(−)-7-[5-[[(1,1′-biphenyl)-4-yl]methoxy]-2-(4-morpholinyl)-3-oxocyclopentyl]-4-heptenoic acid.

WO 00/18405 (Pharmagene) describes the EP4 receptor antagonists AH22921 and AH123848 (which are also described in GB 2 028 805 and U.S. Pat. No. 4,342,756). WO 01/72302 (Pharmagene) describes further EP4 receptor antagonists, for example those described by reference to, and included in the general formula (I) shown on page 8 et seq.

All of these references to EP2 and EP4 receptor antagonists are incorporated herein by reference.

It will be appreciated that one or more EP2 receptor antagonists, or one or more EP4 receptor antagonists, may be administered to the patient. It will also be appreciated that a combination of one or more EP2 or EP4 receptor antagonists may be administered to the patient.

In a preferred embodiment, the patient is administered one or more inhibitors of COX-1 and one or more EP2 or EP4 receptor antagonists. It is particularly preferred that a combination of one or more inhibitors of COX-1 and one or more EP2 or EP4 receptor antagonists is used in the treatment. Treatment with an EP2 and/or EP4 receptor antagonist in the absence of a COX-1 inhibitor may leave further effects of elevated expression of COX-1 untreated, whereas treatment with a COX-1 inhibitor alone may leave the undesirable effects of elevated EP2 and/or EP4 receptor untreated.

Thus, the invention includes the administration to the patient of a combination of one or more inhibitors of COX-1 and one or more EP2 or EP4 receptor antagonists.

The inhibitors of COX-1, or the EP2 or EP4 receptor antagonists, or combinations thereof, are typically administered in an effective amount to combat the neoplastic condition of the cervix. Thus, the treatment agent or agents may be used to alleviate symptoms (ie are used palliatively) or may be used to treat the condition. (By “treatment agent” or “therapeutic agent” we mean the inhibitor of COX-1 or EP″ or EP4 receptor antagonist as the context demands). The method may also be used prophylactically, for example to prevent the development of a neoplastic lesion or to prevent transition from one early stage to a later stage, or by reversing early changes either partially or completely, such as at CIN stages I, II or TH. Thus, the term “treating” specifically includes prophylactic treatment. The treatment agent or agents may be administered by any suitable route, and in any suitable form. It is desirable to administer an amount of the inhibitor of COX-1, or EP2 or EP4 receptor antagonist, or combination thereof, that is effective in alleviating or ameliorating or curing or preventing the neoplastic condition of the cervix.

When more than one of the treatment agents are administered to the patient, it will be appreciated that they can be administered contemporaneously or sequentially. In addition, it may be desirable to administer the agents in different forms or by different routes of administration or to different parts of the patient's body.

A third aspect of the invention provides the use of an inhibitor of COX-1 in the manufacture of a medicament for treating a neoplastic condition of the cervix in a patient. Although the inhibitor of COX-1 may be administered alone, it may desirable if the patient is also administered one or more antagonists of an EP2 or EP4 receptor. By “the patient is also administered” we include that the patient has been, is being or will be administered the said further therapeutic agent or agents.

A fourth aspect of the invention provides the use of an EP2 or EP4 receptor antagonist in the manufacture of a medicament for treating a neoplastic condition of the cervix in a patient. Although the EP2 or EP4 receptor antagonist may be administered alone, it may desirable if the patient is also administered one or more inhibitors of COX-1. By “the patient is also administered” we include that the patient has been, is being or will be administered the said further therapeutic agent or agents.

A fifth aspect of the invention provides the use of any one or more of (a) an inhibitor of COX-1 and (b) an EP2 or EP4 receptor antagonist for treating a neoplastic condition of the cervix.

While it is possible for a compound of the invention to be administered alone or in a “naked” form, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

COX-1 inhibitors are known to have adverse effects in the stomach where, for example, under certain conditions they may be involved in forming ulcers. It is preferred that if the COX-1 inhibitor is administered orally that it is present in formulation which is designed to limit such possible adverse effects. Such formulations are well known in the art and include, for example, enteric coated tablets wherein the coating is resistant to the acid conditions in the stomach (and so the COX-1 inhibitor is not released in the stomach) but is not resistant to the alkali conditions in the small intestines where the COX-1 inhibitor may be released.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier. Buccal administration is also preferred.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

Certain EP2 and EP4 receptor antagonists are proteins or peptides. Proteins and peptides may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The protein and peptide can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of proteins and peptides. A device which delivers a pulsed electric field to cells increases the permeability of the cell membranes to the drug, resulting in a significant enhancement of intracellular drug delivery.

Proteins and peptides can be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and peptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The EP2 or EP4 receptor antagonist is delivered over time as the biopolymers dissolve.

Protein and peptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Proteins and polypeptides can be introduced to cells by “Trojan peptides”. These are a class of polypeptides called penetratins which have translocating properties and are capable of carrying hydrophilic compounds across, the plasma membrane. This system allows direct targeting of oligopeptides to the cytoplasm and nucleus, and may be non-cell type specific and highly efficient. See Derossi et al (1998), Trends Cell Biol 8, 84-87.

The treatment agent (or combination thereof) is administered at a dose (or in multiple doses) which produces a beneficial therapeutic effect in the patient. Suitable doses may be determined by the physician. The dose to be administered is determined upon age, body weight, mode of administration, duration of the treatment, and pharmacokinetic and toxicological properties of the treatment agents.

It is preferred if the treatment agent (or combination thereof) is administered orally. It is further preferred if the treatment agent (or combination thereof) is administered to the female reproductive system. For example, the treatment agent may suitably be administered intravaginally using, for example, a gel or cream or vaginal ring or tampon. The treatment agent (or combination thereof) may also advantageously be administered using an intrauterine device, although this route of administration is less preferred for carcinoma in situ and invasive carcinoma (but may be used for treating at CIN grades I II or III).

Typically, the gel or cream is one which is formulated for administration to the vagina. It may be oil based or water based. Typically, the treatment agent (or combination thereof) is present in the cream or gel in a sufficient concentration so that an effective amount is administered in a single (or in repeated) application.

Typically, the vaginal ring comprises a polymer which formed into a “doughnut” shape which fits within the vagina. The treatment agent (or combination thereof) is present within the polymer, typically as a core, which may dissipate through the polymer and into the vagina and/or cervix in a controlled fashion. Vaginal rings are known in the art. The vaginal ring may be disposable; alternatively, the vaginal ring may be used over a time interval of around three months to one year, during which time sufficient treatment agent (or combination thereof) is released to have a beneficial effect over that period of time. It will be appreciated that the polymer from which the ring is made, the size and shaper of the ring and the content of antagonist, as well as other parameters, may be selected by reference to how long the ring is in situ in the vagina.

Typically, the tampon is impregnated with the treatment agent (or combination thereof) and that a sufficient amount of the treatment agent (or combination thereof) is present in the tampon bearing in mind that the tampon is generally used for a single day.

Typically, the intrauterine device is for placing in the uterus over extended periods of time, such as between one and five years. Typically, the intrauterine device comprises a plastic fame, often in the shape of a “T” and contains sufficient treatment agent (or combination thereof) to be released over the period of use. The treatment agent (or combination thereof) is generally present within or encompassed by a slow-release polymer which forms part of the device, such as in the form of a “sausage” of antagonist which wraps around the long arm of the “T” which is typically covered with a controlled-release membrane. Intrauterine devices are known in the art.

A sixth aspect of the invention provides a therapeutic system for treating a neoplastic condition of the cervix the system comprising a combination of any two or more of (a) an inhibitor of COX-1 and (b) an EP2 or EP4 receptor antagonist. The particular treatment agents in the therapeutic system may be selected according to the preferences given above. The therapeutic system may be purchased ready to use, or it may be assembled by the pharmacist or physician.

A seventh aspect of the invention provides a therapeutic system for treating a neoplastic condition of the cervix the system comprising any one or more of (a) an inhibitor of COX-1 and (b) an EP2 or EP4 receptor antagonist adapted for delivery to the cervix. Suitable adaptations for delivery of the treatment agent to the cervix are described above.

The invention will now be described in more detail with reference to the following non-limiting Examples and Figures wherein:

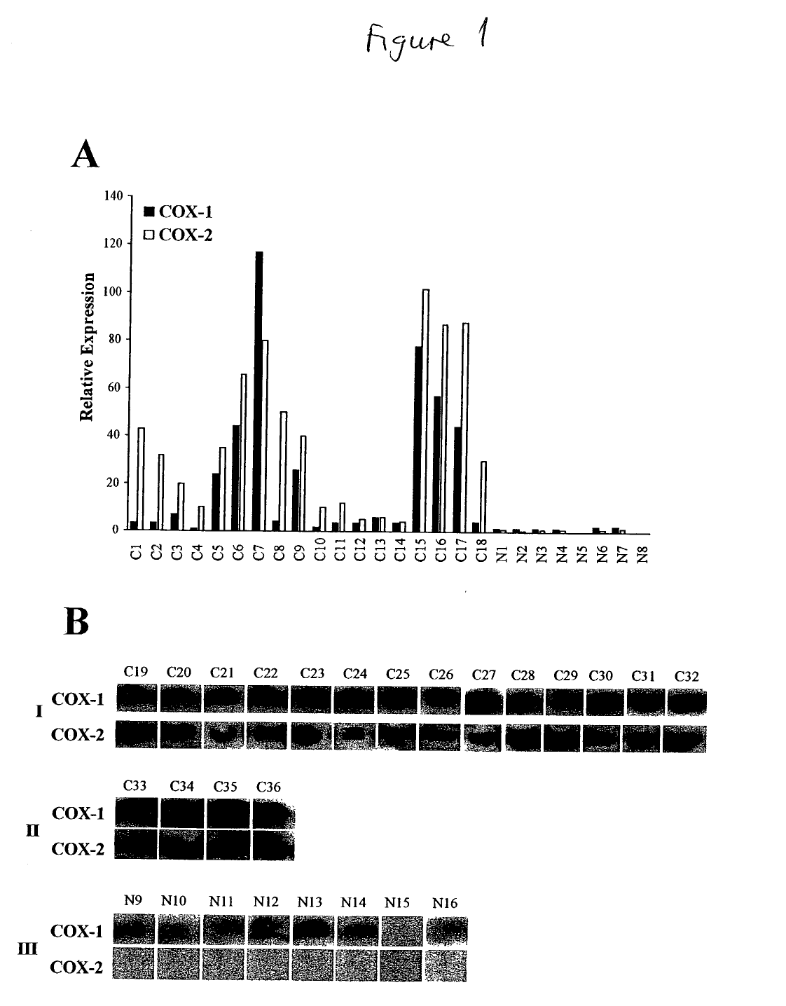

FIG. 1. (A) Relative expression of COX-1 and COX-2 RNA in cervical squamous cell carcinoma (C1-C14), adenocarcinoma (C15-C18) and normal cervix (N1-N8) as determined by real-tine quantitative RT-PCR. (B) Western blot analysis of 50 μg total protein isolated from human cervical carcinoma tissue. The proteins were loaded onto a 10% SD S-gel, electophoresed and subsequently transferred to PVDF membrane. The immunoblot was probed with antibody raised against the C-terminus of human COX-1 or COX-2. A specific band of approximately 72 kDa was detected in all squamous cell carcinomas (panel I; C19-C32) and adenocarcinomas (panel II; C33-C36). Basal COX-1 expression was detected in 7/8 normal cervices. No COX-2 expression was detected in normal cervical tissue (panel III, N9-N16).

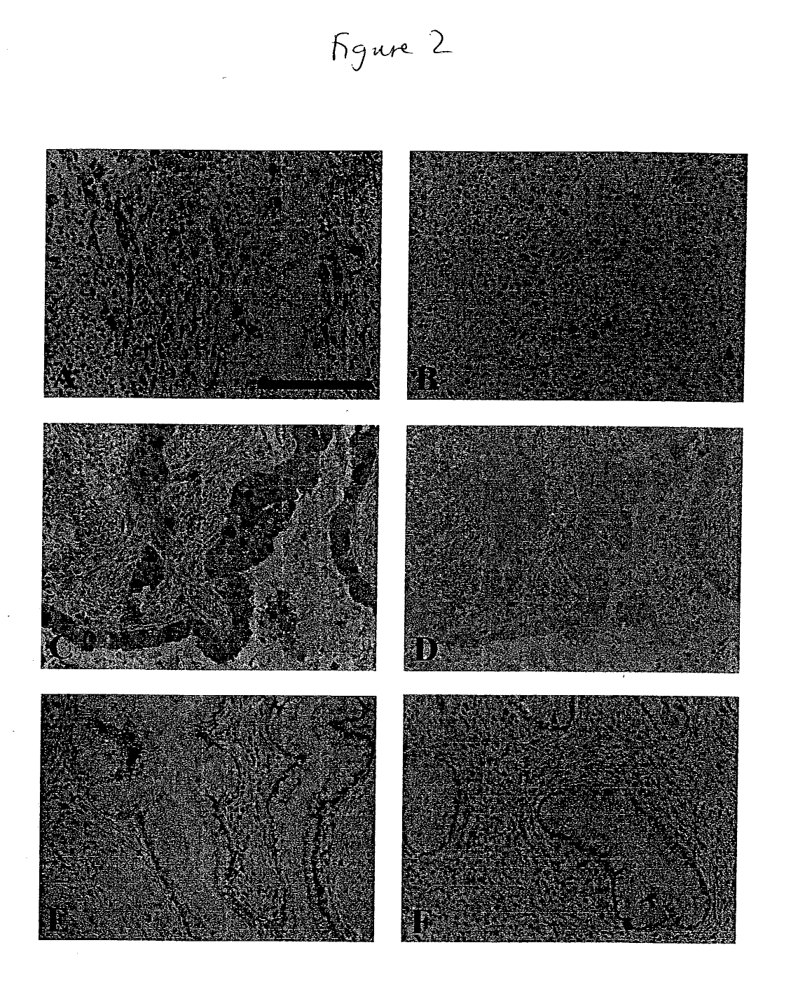

FIG. 2. Localisation of COX-1 expression in epithelial cells of squamous cell carcinomas and columnar and glandular epithelium of adenocarcinomas (A and C respectively). Minimal COX-1 signal was detected in normal cervical tissue (E). Sections that were stained with pre-adsorbed COX-1 sera are shown in B, D and F for squamous cell carcinoma, adenocarcinoma and normal cervix respectively (negative controls). Scale bar is 100 gm.

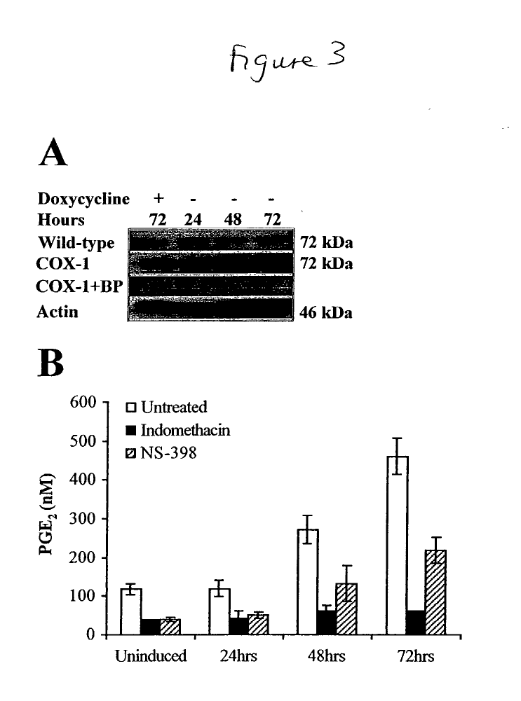

FIG. 3. (A) Western blot analysis of 20 μg total protein isolated from wild-type HeLa Tet-Off and HeLa COX-1 Tet-Off cells grown for 24, 48 and 72 hrs respectively in the absence of DOX. In parallel control uninduced HeLa COX-1 Tet-Off and wild-type HeLa Tet-Off cells were maintained for 72 hrs under the same conditions supplemented daily with DOX to a final concentration of 1 μg/ml. The proteins were loaded onto a 4-20% SDS-gel, electrophoresed and subsequently transferred to PVDF membrane. The immunoblot was probed with antibody raised against the C-terminus of human COX-1. A specific band of approximately 72 kDa was detected. No immunoreactivity was detected by pre-adsorbing the antibody with the blocking peptide (BP). COX-1 was normalised for protein loading against β-actin on the same blot. (13) The functionality of the transfected COX-1 cDNA was assessed by ELISA, by measuring PGE₂ secretion into the culture medium following COX-1 induction in the presence or absence of the COX-enzyme inhibitor indomethacin, and treatment of HeLa cells with 5 μg/ml arachidonic acid.

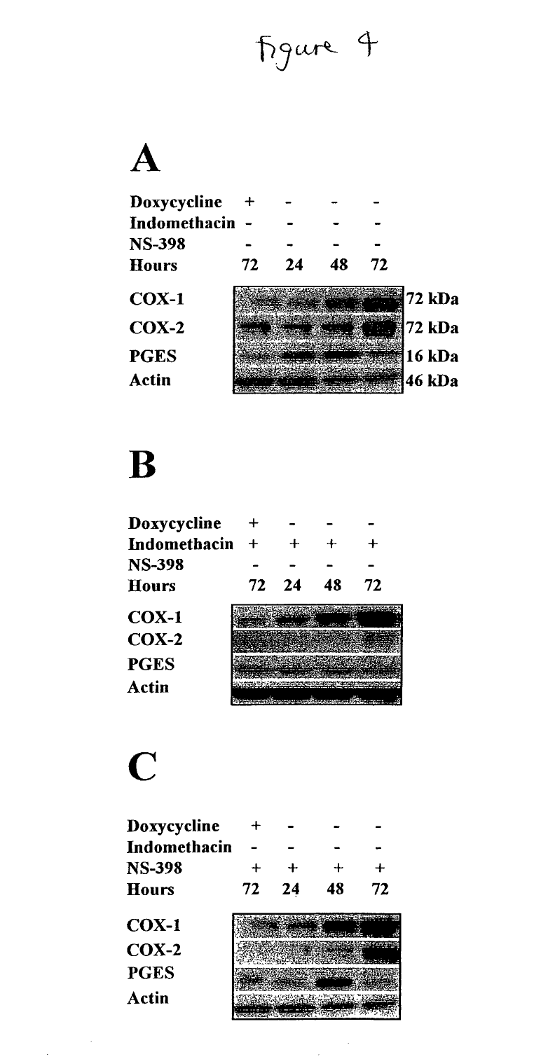

FIG. 4. Western blot analysis of 20 μg of total clarified cell lysate isolated from HeLa COX-1 Tet-Off cells grown for 72 hrs in the presence of DOX (uninduced) or 24, 48 and 72 hrs respectively in the absence of DOX to induce COX-1 expression. (A) Expression of COX-2 and PGES was induced coincident with COX-1 overexpression in HeLa cells. (B) Co-treatment of HeLa cells with indomethacin abolished the COX-1-mediated up-regulation of COX-2 and PGES. (C) Partial inhibition of the COX-1-mediated up-regulation of COX-2 and PGES expression was observed following co-treatment with the selective COX-2 inhibitor NS-398. Proteins were normalised for loading against A-actin on the same blot.

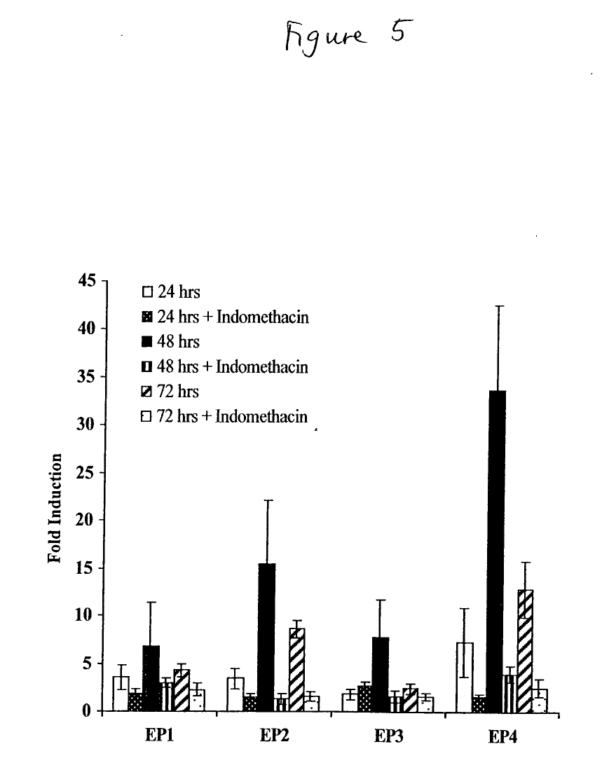

FIG. 5. Fold induction of expression of PGE₂ receptors (EP1-EP4) in HeLa COX-1 Tet-Off cells as determined by real-time quantitative RT-PCR. COX-1 expression was induced for 24, 48 and 72 hrs in the presence or absence of the COX enzyme inhibitor indomethacin. Fold induction was determined by dividing the relative expression in induced cells by the relative expression in uninduced cells.

FIG. 6. cAMP levels in HeLa COX-1 Tet-Off following treatment with 0 or 300 nM PGE₂. Cells were either maintained with 1 μg/ml DOX (uninduced) or induced by incubation in culture medium without DOX for 48 hrs in the presence or absence of the COX-enzyme inhibitor indomethacin.

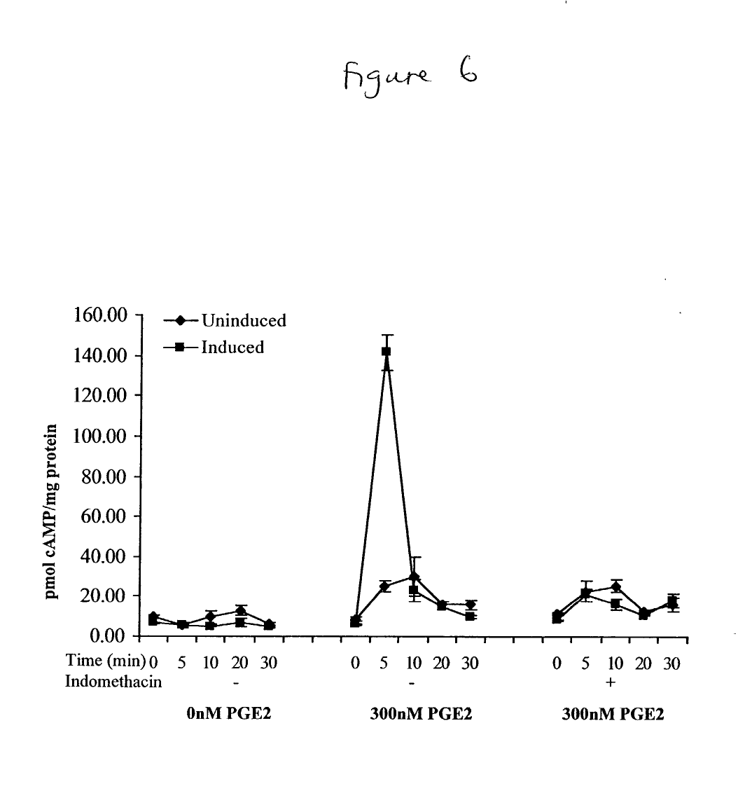

FIG. 7. Western blot analysis of 20 μg of total clarified cell lysate isolated from HeLa COX-1 Tet-Off cells grown for 72 hrs in the presence of DOX or 24, 48 and 72 hrs in the absence of DOX to induce COX-1 expression. (A) Immunoblot of bFGF expression following DOX withdrawal from the culture medium. bFGF expression was induced coincident with COX-1 overexpression. Upregulated bFGF expression was abolished by indomethacin and partially inhibited by NS-398. (B) Immunoblot of VEGF expression following DOX withdrawal from the culture medium. VEGF was induced after 72 hrs of COX-1 overexpression. Up-regulated VEGF expression was abolished by indormethacin and partially inhibited by NS-398. (C) Immunoblot of Ang-1 expression following DOX withdrawal from, the culture medium. Ang-1 was induced coincident with COX-1 overexpression after 48 hrs. Up-regulated Ang-1 expression was abolished by indomethacin and partially inhibited by NS-398. (D) Immunoblot of Ang-2 expression following DOX withdrawal from the culture medium. Ang-1 was induced after 48 hrs of COX-1 overexpression. Up-regulated Ang-2 expression was abolished by indomethacin and partially inhibited by NS-398. Proteins were normalised for loading against (3-actin.

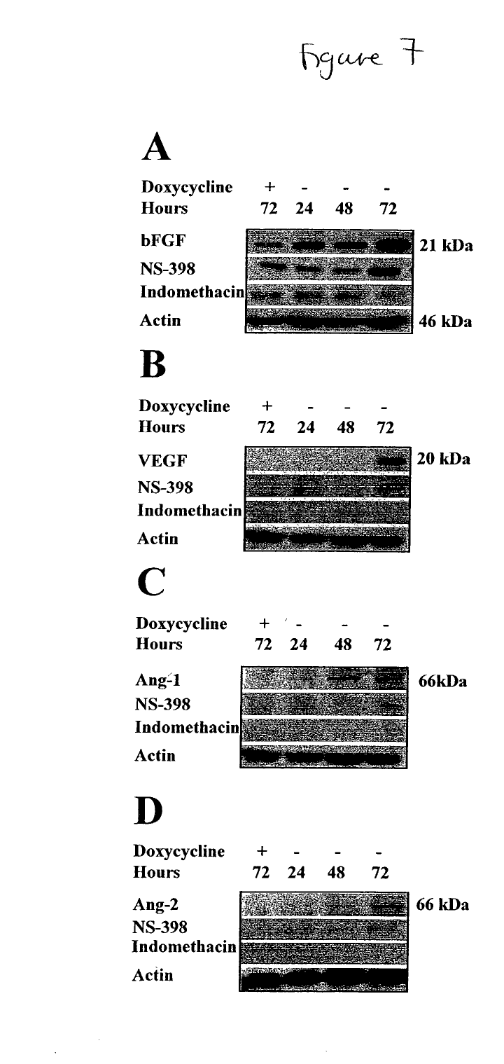

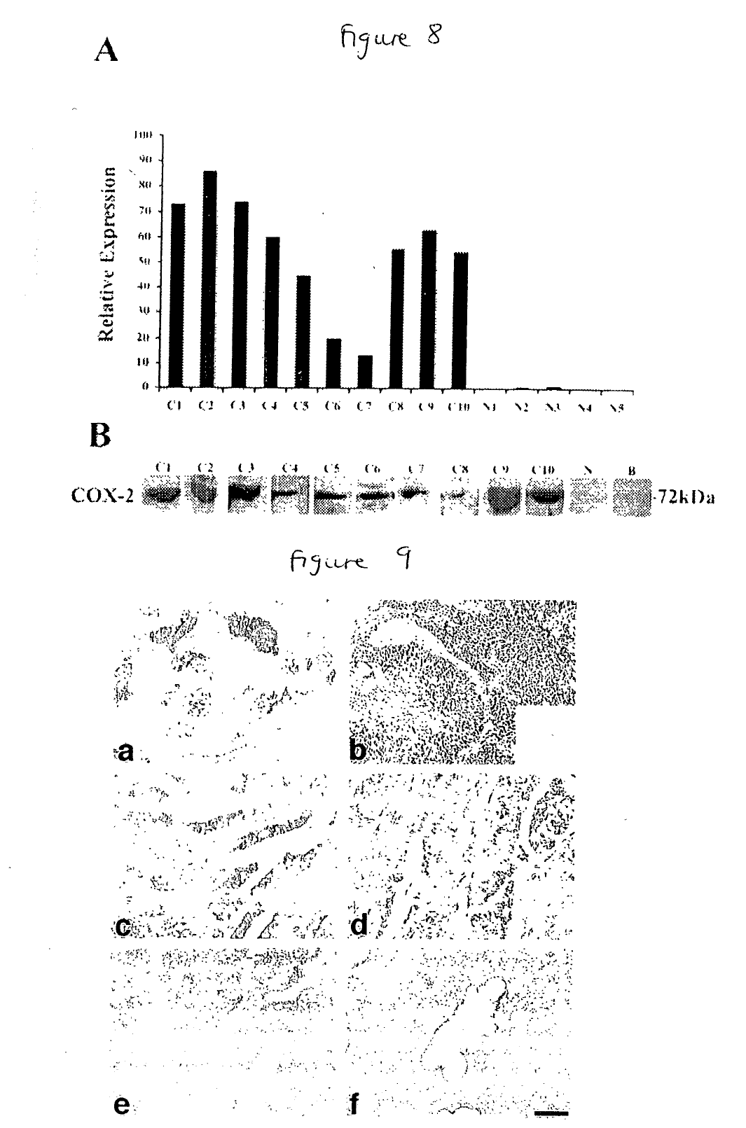

FIG. 8. (A) Relative expression of COX-2 RNA in cervical squamous cell carcinoma (C1-C8), adenocarcinoma (C9 and C10) and normal cervix (N1-N5) as determined by real-time quantitative RT-PCR. (B) Western blot analysis of 100 μg total protein isolated from human cervical carcinoma tissue. The proteins were loaded onto a 10% SDS-gel, electrophoresed and subsequently transferred to PVDF membrane. The immunoblot was probed with antibody raised against the C-terminus of human COX-2. A specific band of approximately 72 kDa was detected in all squamous (C1-C8) and adenocarcinoma (C9 and C10). No signal was detected in normal cervical tissue (N) (a representative sample is shown). Moreover, preadsorbing the antibody with the blocking peptide (B) abolished the COX-2 signal in all carcinoma samples (a representative sample is shown).

FIG. 9. COX-2 expression and PGE₂ synthesis (Figures a and b respectively) are detected in epithelial cells of squamous cell carcinoma and columnar and glandular epithelium of adenocarcinomas (Figures c and d respectively). Minimal COX-2 and PGE₂ signal was detected in normal cervical tissue (Figures e and f respectively). Figures g and h respectively are representative sections of adenocarcinoma incubated with COX-2 antibody preadsorbed with the blocking peptide (COX-2 negative control) and non-immune rabbit serum (PGE₂ negative control). Scale bar is 100 μm.

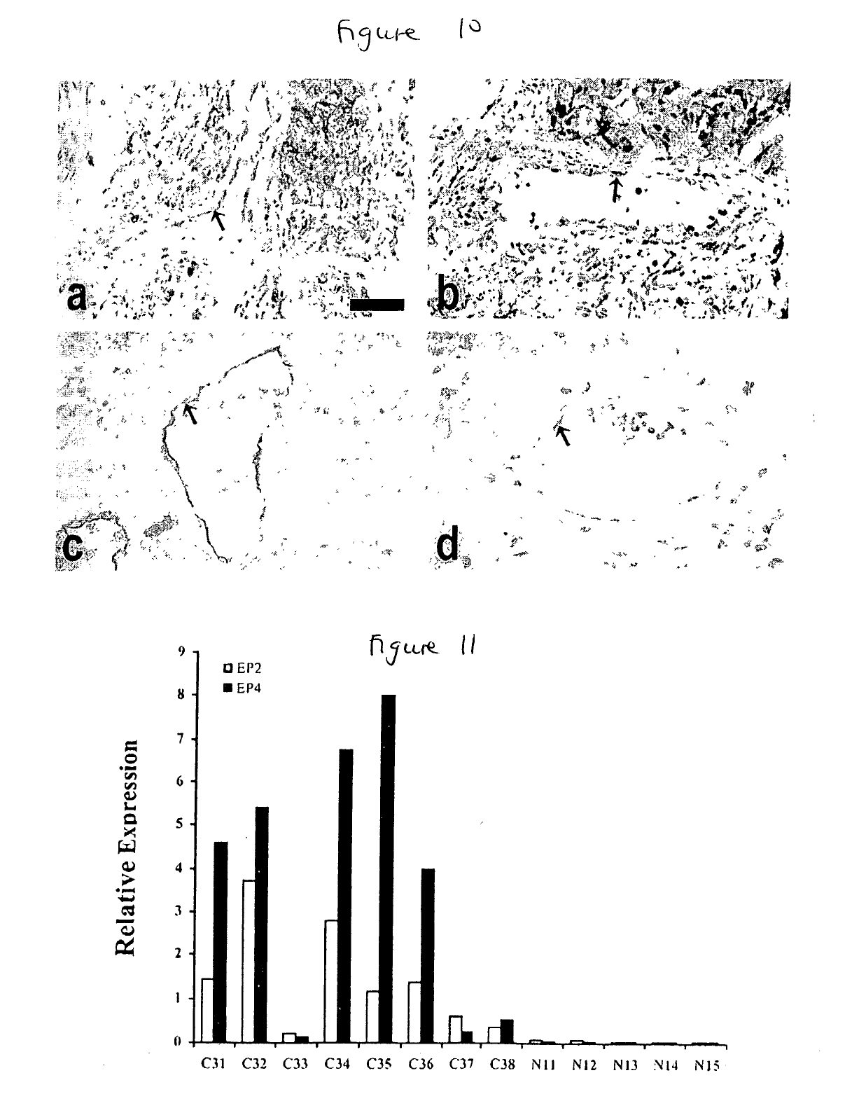

FIG. 10. COX-2 (Figure a) expression and PGE₂ (Figure b) synthesis are detected in endothelial cells (arrowed) of all carcinoma tissues. Vascular endothelial cells in cervical cancer tissues were localised using antibodies raised against the human CD34 endothelial cell marker (Figure c). Figure d is a representative section incubated with non-immune goat serum (CD34 negative control). Scale bar is 50 μm.

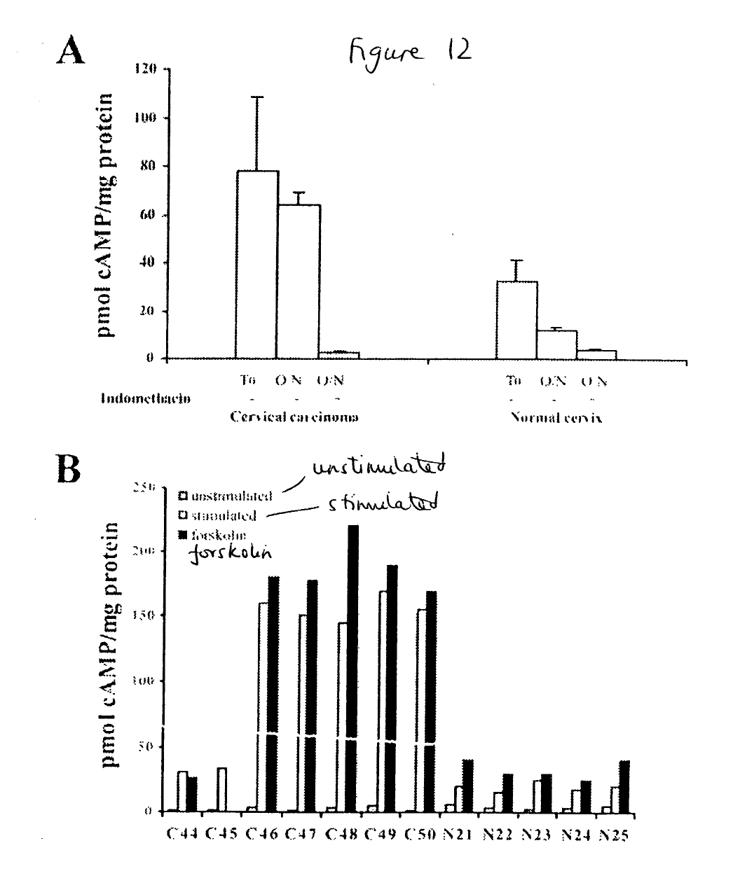

FIG. 11. Relative expression of EP2 (empty bars) and EP4 (solid bars) receptors in cervical squamous cell carcinoma (C1-C7), adenocarcinoma (C8) and normal cervix (N1-N5) as determined by real-time quantitative RT-PCR

FIG. 12. (A) Basal cAMP levels (pmol cAMP/mg protein) in cervical tissues (mean±SEM of n=7 squamous cell carcinomas and n=5 normal cervix). Basal cAMP levels were determined shortly after biopsy (T₀) and after overnight (O/N) culture in the absence (−) or presence (+) of indomethacin. (B) cAMP response (pmol cAMP/mg protein) in squamous cell carcinoma (C1-C6), adenocarcinoma (C7) and normal cervix (N1-N5). Cervical tissues were treated with indomethacin overnight and either stimulated with 300 nM PGE₂ (empty bars) or 50 μM forskolin (solid bars; positive control) or left unstimulated (grey bars).

### EXAMPLE 1

### Cyclooxygenase-1 is Up-Regulated in Cervical Carcinomas: Autocrine/Paracrine Regulation of Cyclooxygenase-2, PGE Receptors and Angiogenic Factors by Cyclooxygenase-1

This study was designed to investigate the expression and molecular signalling of cyclooxygenase-1 (COX-1) in cervical carcinomas. Real-time quantitative RT-PCR and Western blot analysis confirmed enhanced expression of COX-1 RNA and protein in squamous cell carcinomas and adenocarcinoma of the cervix. COX-1 expression in all carcinoma tissues was associated with enhanced expression of COX-2 RNA and protein. The site of COX-1 expression was localised by immunohistochemistry to the neoplastic epithelial cells in all squamous cell carcinomas and adenocarcinomas studied. Minimal COX-1 immunoreactivity was detected in normal cervix. To explore events associated with COX-1 up-regulation, we developed a doxycycline (DOX)-regulated expression system in HeLa (cervical carcinoma) cells. Overexpression of COX-1 in HeLa cells resulted in induced expression of cyclooxygenase-2 (COX-2) and prostaglandin E synthase (PGES) concomitant with increased prostaglandin E2 (PGE₂) synthesis. Treatment of HeLa cells overexpressing COX-1 with the dual COX-enzyme inhibitor indomethacin or selective COX-2 inhibitor NS-398 significantly reduced PGE₂ synthesis. Indomethacin, but not NS-398, treatment abolished the up-regulation of expression of COX-2 and PGES in HeLa cells suggesting that the observed up-regulation of COX-2 and PGES was mediated by COX-1-enzyme products. In order to assess whether enhanced PGE₂ synthesis, following COX-1 induction, would act in an autocrine/paracrine manner, we investigated the effect of COX-1 on the expression of the different isoforms of PGE receptors (EP1-4). We found that the cAMP-linked PGE₂ receptors were significantly up-regulated by COX-1 overexpression coincident with enhanced cAMP responsiveness of these cells to exogenous PGE₂ ligand. Finally, overexpression of COX-1 was associated with enhanced expression of the angiogenic factors; basic fibroblast growth factor (bFGF), vascular endothelial growth factor (VEGF), angiopoietin-1 (Ang-1) and angiopoietin-2 (Ang-2). This up-regulation of angiogenic factor expression was abolished by indomethacin and partially reduced by NS-398. These data indicate that COX-1 up-regulation modulates the expression of factors that may act in an autocrine/paracrine manner to enhance and sustain tumourigenesis in neoplastic cervical epithelial cells. It is likely that similar mechanisms may act in vivo to modulate tumourigenesis of cervical carcinomas.

Introduction

Uterine cervical cancer is considered an important clinical problem in developing countries, with high incidence of invasive disease reported for South African women (1). Three histological categories of epithelial tumours of the cervix are recognised by the World Health Organisation (2). These are squamous cell carcinoma, adenocarcinoma and other less common types of epithelial tumours. The most common histological type of cervical carcinoma is squamous cell carcinoma that accounts for 60%-80% of all cervical cancers. Adenocarcinoma accounts for less than 20% of invasive cervical carcinomas. Numerous studies have-demonstrated that epithelial tumours may be regulated by cyclooxygenase (COX)-enzyme products (3-7). Two distinct isoforms of the COX enzyme, COX-1 and COX-2 have been reported (8-10). The relative contributions of COX-1 and/or COX-2-derived products in mediating events associated with cervical neoplasia remain to be elucidated. COX-1 expression is considered to be constitutive and generate prostaglandins for normal physiological functions (4, 11, 12). Transcription of COX-2 RNA and protein are up-regulated in several epithelial carcinomas (3, 12-15) including carcinomas of the cervix (16-18). This has prompted the suggestion that the increased level of prostaglandins and other eicosanoids present in cancer tissue is a consequence of induced COX-2. More recently however, it has been demonstrated that both COX isoforms are inducible. In some cell types, including pulmonary artery endothelial cells, COX-1 levels are induced during differentiation (19, 20). COX-1 expression can be induced in vitro by vascular endothelial growth factor (VEGF) (21), arachidonic acid, forskolin, dibutryl-cAMP and prostaglandin E2 (PGE₂) (22). In addition, elevated COX-1 expression has been reported in mouse lung tumours (23), human breast cancer (24) and human prostate carcinoma (25). These data suggest that both COX enzymes and/or their products may function in promoting and maintaining the neoplastic state. COX catalyse the double oxygenation and reduction of arachidonic acid, following its release from membrane glycerophospholipids by phospholipase A₂, to the intermediate form prostaglandin H₂. This intermediate serves as the substrate for terminal prostanoid synthases, which produce their specific prostaglandins such as PGE₂ being synthesised by prostaglandin E synthase (PGES) (26-28). PGE₂ has been shown to stimulate gene transcription (29), influence mitogenesis of normal human bone cells (30) and promote growth and metastasis of tumours (31). More recently, enhanced synthesis of PGE₂ resulting from up-regulated COX-2 has been shown to induce malignant change in epithelial cells through immunosuppression (32), inhibiting apoptosis (13), increasing metastatic potential of epithelial cells (6) and promoting angiogenesis (33, 34). Two segregated biosynthetic pathways have been described for PGE₂ biosynthesis. These pathways synthesise PGE₂ via PGES functionally and preferentially coupled with either COX-1 or COX-2 (27). The biological actions of PGE₂ have been attributed to its interaction with G-protein-coupled seven-Transmembrane-domain receptors (GPCRs), which belong to the rhodopsin superfamily of serpentine receptors (35). Four main sub-types of PGE₂ receptors have been identified (EP₁, EP₂, EP₃, EP₄) which utilise alternate and in some cases opposing intracellular pathways (36). Most studies have focussed on neoplastic events associated with COX-enzyme products as a consequence of COX-2 overexpression. In this study, we investigated (a) COX-1 expression and localisation in cervical squamous cell carcinomas and adenocarcinomas compared with normal cervical tissue, and (b) a possible autocrine/paracrine role for COX-1-enzyme products in regulating the expression of COX-2, PGES, PGE₂ receptors and angiogenic factors in cervical epithelial carcinoma cells using an inducible expression system.

Materials and Methods

Materials

The following antibodies used for Western blotting were purchased from Santa Cruz Biotechnology (Autogenbioclear, Wiltshire, UK): COX-1 goat polyclonal (sc-1752), COX-2 goat polyclonal (sc-1745), bFGF goat polyclonal (sc-1360), VEGF rabbit polyclonal (sc-152), Ang-1 goat polyclonal (sc-6319), Ang-2 goat polyclonal (sc-7016), β-actin goat polyclonal (sc-1616) as well as the COX-1 and COX-2 blocking peptides (sc-1752p and sc-1745p). The PGES antibody raised against the microsomal glutathione-dependent inducible PGES (27) was purchased from Caymen Chemical Co. (Caymen Chemical Co., Cheshire, UK); anti-goat-alkaline phosphatase (AP), anti-rabbit-AP, cloning cylinders, G418, hygromycin (Hyg), doxycycline (DOX) and indomethacin were purchased from Sigma (Sigma Chemical Company, Dorset, UK). Samples and synthetic standards for the PGE₂ ELISA were purchased from Applied Therapeutics (Applied Therapeutics, Paisley, UK) and NS-398 was purchased from Calbiochem (Calbiochem, Nottingham, UK). HeLa Tet-Off cells and Tet-system-approved foetal bovine serum were purchased from Clontech (Clontech, Hampshire, UK). Dulbecco's modified Eagle's medium nutrient mixture F-12 was purchased from Life Technologies (Gibco, Life Technologies, Paisley, UK) and penicillin-streptomycin was purchased from PAA (PAA Laboratories Ltd., Middlesex, UK). ECF chemiluminescence system was purchased from Amersham (Amersham, Little Chalfont, Bucks, UK).

Tissue Collection and Processing

Cervical specimens were obtained at the time of surgery/biopsy from patients that were attending the Gynaecologic Oncology Clinic at Groote Schuur Hospital, Cape Town and that had previously been diagnosed with invasive carcinoma of the cervix. Punch biopsies were taken from the lesion by an experienced Gynaecologist with a special interest in oncology. A portion of the biopsy was excised and fixed in formalin followed by paraffin wax-embedding for histopathological typing. The remaining portion was snap frozen in either dry ice or liquid nitrogen and stored at −70° C. for RT-PCR and Western blot analysis. The extent of invasiveness of carcinoma biopsies (C1-C58) is represented in Table 1. Histologically normal cervical samples (N1-N21) were obtained from patients undergoing Wertheims hysterectomy for non-malignant conditions. Pathological typing was defined according to the International Federation of Obstetricians and Gynaecologists (37) staging up-on physical examination. The ages of the patients ranged from 29 years to 80 years with a median age of 50 years, The study was approved by the University of Cape Town Research Ethics Committee and informed consent was obtained from all patients before tissue collection.

Cell Culture

HeLa Tet-Off cells containing the regulatory plasmid (pTet-Off) were routinely maintained in Dulbecco's modified Eagle's medium nutrient mixture F-12 with glutamax-1 and pyridoxine, supplemented with 10% foetal bovine serum, 100 μg/ml G418 and 1% antibiotics (stock 500 IU/ml penicillin and 500 μg/ml streptomycin) at 37° C. and 5% CO₂ (v/v).

Cell Transfections

The Tet-Off expression system we used was developed by Gossen and co-workers (38) to deliver doxycycline-regulated expression based on the high specificity of the Escherichia coli tet repressor-operator-doxycycline interaction. In the Tet-Off expression system each clonal cell line is used as its own control (cells cultured in the presence of DOX) and the overexpression of the integrated target gene is modulated solely by removing DOX from the culture medium. This eliminates the need for a control clonal cell line transfected with vector alone (as used with constitutive stable expression systems) thereby overcoming the inherent variation that arises from different sites of integration of DNA between different clones. HeLa Tet-Off cells containing the pTet-Off vector stably transfected and constitutively expressing the tetracycline-controlled transactivator tTA (composed of a fusion of the TetR and VP16 activation domain) were purchased from Clontech. The pBS(SK−)/PSHI cDNA containing the full length COX-1 gene (kindly supplied by Dr Stephen Prescott, University of Utah, Salt Lake City, Utah) was used as the template plasmid. The response plasmid pTRE2 (containing the minimal cytomegalovirus promoter containing Tet-operator sequences cloned upstream of the cDNA to be expressed) and the plasmid for antibiotic selection (pTK-hygromycin) for use with the Tet-Off system were purchased from Clontech. The COX-1 gene was excised from the template plasmid and ligated at the Bam H1 site of the pTRE2 vector. The orientation of the insert was verified by dideoxy DNA sequencing using the sequence specific primers: 5′-CGCCTGGAGACGCCATCC-3′ (SEQ ID No 8) and 5′-CCACACCTCCCCCTGAAC-3′ (SEQ ID No 9) (Clontech). Cells were plated in 12 well dishes in complete medium containing 10 μg/ml G418 per well and were allowed to attach and grow overnight. The pTRE2 vector containing the COX-1 gene (2 μg) was co-transfected with pTK-hygromycin (0.1 μg, which contains the hygromycin gene under control of the minimal thymidine kinase (TK) promoter) into the HeLa Tet-Off cell line at about 80% confluency using pfx-5 (Invitrogen, De Schelp, Netherlands) diluted in Optimem (Gibco). Cells were incubated for 4 hrs at 37° C. in 5% humidified CO₂. Thereafter the medium was replaced with fresh complete medium containing no G418. Cells were allowed to grow for 72 hrs. Transfected cells were then seeded together with wild-type cells. Clones were selected against 200 μg/ml hygromycin in the presence of 1 μg/ml DOX. At least 50 hygromycin-resistant clones were picked using cloning cylinders. Clones were allowed to grow under continuous selection with hygromycin in the presence of DOX and then screened for the ability to express COX-1 in the presence and absence of DOX by immunoblot analysis. Three clones with the greatest inducible overexpression of COX-1 (clone 1.2, 2.2 and 3.1) were selected for further experiments. All clones were characterised and exhibited identical phenotypic and biochemical alterations. The results of our studies using the COX-1 clone 1.2 are presented here. Similar reproducible results were obtained using clone 2.2 and 3.1. Unless otherwise stated, all clones were maintained uninduced in 1 μg/ml DOX, 200 μg/ml hygromycin and 100 μg/ml G418. COX-inhibition studies were conducted by growing cells in medium containing 3 μg/ml indomethacin or 10 μM NS-398.

Real-Time Quantitative RT-PCR

Real-time quantitative RT-PCR was performed to determine COX-1 and COX-2 expression in cervical carcinoma biopsies and normal cervical tissue as well as to assess the effect of COX-1 overexpression on expression of the different isoforms of PGE₂ receptors (EP1, EP2, EP3 and EP4) in HeLa Tet-Off cells. RNA samples were extracted from cervical tissue (squamous cell carcinomas, C1-C14; adenocarcinomas, C15-C18 and normal cervix, N1-N8) using Tri-Reagent (Sigma) as per the manufacturer's instruction. To determine the effect of COX-1 overexpression on expression of EP receptors, cells (2×10⁵) were seeded in 6 well plates, allowed to attach and grow overnight in the presence of DOX. The following day, the cells were synchronised by incubating with serum-free medium for 24 hrs. Thereafter, the medium was replaced with fresh complete medium and COX-1 overexpression was induced by growing cells in medium containing no DOX. Control cells were maintained in DOX. Cells were harvested after 24, 48 and 72 hrs with 1 ml/well Tri-Reagent (Sigma) as per the manufacturer's protocol. RNA samples were reverse transcribed using MgCl₂ (5.5 mM), dNTPs (0.5 mM each), random hexamers (1.25 μM), oligo-dT (1.25 μM), RNAase inhibitor (0.4 U/μl) and multiscribe reverse transcriptase (1.25U/μl; all from PE Biosystems, Warrington, UK). The mix was aliquoted into individual tubes (16 μl/tube) and template RNA was added (4 μl/tube of 250 ng/μl RNA). Samples were incubated for 60 mins at 25° C., 45 mins at 48° C. and then at 95° C. for 5 mins. A reaction mix was made containing Taqman buffer (5.5 mM MgCl₂, 200 μM dATP, 200 μM dCTP, 200 μM dGTP, 400 μM dUTP), ribosomal 18S forward and reverse primers and probe (all at 50 nM), forward and reverse primers for COX-1, COX-2, EP1, EP2, EP3-or EP4 receptor (300 nM), COX-1, COX-2, EP1, EP2, EP3 or EP4 receptor probe (200 nM), AmpErase UNG (0.01 U/μl) and AmpliTaq Gold DNA Polymerase (0.025 U/μl; all from PE Biosystems). A volume of 481 μl of reaction mix was aliquoted into separate tubes for each cDNA sample and 2 μl/replicate of cDNA was added. After mixing, 23 μl of sample were added to the wells on a PCR plate. Each sample was added in duplicate. A no template control (containing water) was included in triplicate. Wells were sealed with optical caps and the PCR reaction run on an ABI Prism 7700 using standard conditions. COX-1, COX-2 and EP receptor primers and probe for quantitative PCR were designed using the PRIMER express program (PE Biosystems). The sequence of the COX-1 primers and probe were as follows: Forward: 5′-TGT TCG GTG TCC AGT TCC AAT A-3′ (SEQ ID No 10); Reverse: 5′-ACC TTG AAG GAG TCA GGC ATG AG-3′ (SEQ ID No 11); Probe (FAM labelled): 5′-CGC AAC CGC ATT GCC ATG GAG T-3′ (SEQ ID No 12). The sequence of the COX-2 primers and probe were as follows: Forward: 5′-CCT TCC TCC TOT GCC TGA TG-3′ (SEQ ID No 13); Reverse, 5′-ACA ATC TCA TTT GAA TCA GGA AGC T-3′ (SEQ ID No 14); Probe (FAM labelled): 5′-TGC CCG ACT CCC TTG GOT GTC A-3′ (SEQ ID No 15). The sequence of the EP1 receptor primers and probe were as follows: Forward: 5′-AGA TGG TGG GCC AGC TTG T-3′ (SEQ ID No 16); Reverse: 5′-GCC ACC AAC ACC AGC ATT G-3′ (SEQ ID No 17); Probe (FAM labelled): 5′-CAG CAG ATG CAC GAC ACC ACC ATG-3′ (SEQ ID No 18). The sequence of the EP2 receptor primers and probe were as follows; Forward: 5′-GAC CGC TTA CCT GCA GCT GTA C-3′ (SEQ ID No 19); Reverse: 5′-TGA AGT TGC AGG CGA GCA-3′ (SEQ ID No 20); Probe (FAM labelled): 5′-CCA CCC TOC TGC TGC TTC TCA TTG TCT-3′ (SEQ ID No 21). The sequence of the EP3 receptor primers and probe were as follows; Forward: 5′-GAC GGC CAT TCA GCT TAT GG-3′ (SEQ ID No 22); Reverse: 5′-TTG AAG ATC ATT TTC AAC ATC ATT ATC A-3′ (SEQ ID No 23); Probe (FAM Labelled): 5′ CTG TCG OTC TGC TGG TCT CCG CTC-3′ (SEQ ID No 24). The sequence of the EP4 receptor primers and probe were as follows; Forward: 5′-ACG CCG CCT ACT CCT ACA TG-3′ (SEQ ID No 25); Reverse: 5′-AGA GGA CGG TGG CGA GAA T-3′ (SEQ 11) No 26); Probe (FAM labelled); 5′-ACG CGG GCT TCA GCT CCT TCC T-3′ (SEQ ID No 27). The ribosomal 18S primers and probe sequences were as follows; Forward: 5′-CGG CTA CCA CAT CCA AGG AA-3′ (SEQ ID No 28); Reverse: 5′-GCT GGA ATT ACC GCG GCT-3′ (SEQ ID No 29); Probe (VIC labelled): 5′-TGC TGG CAC CAG ACT TGC CCT C-3′ (SEQ ID No 30). Expression of COX-1 and EP receptors were normalised to RNA loading for each sample using the 18S ribosomal RNA as an internal standard. Relative COX-1 and COX-2 expression in carcinoma tissue was calculated by dividing the expression in carcinoma tissue by the expression in normal cervix. Relative expression of EP receptors was calculated, from 3 independent experiments, by dividing the expression in induced cells by expression in uninduced cells. The data are presented as mean±SEM.

Protein Extraction

Tissue

COX-1 and COX-2 protein expression in cervical carcinomas and normal cervix was assessed by Western blotting. Proteins were extracted from cervical tissue (squamous cell carcinomas, C19-032; adenocarcinomas C33-C36 and normal cervix, N9-N16) by homogenisation in protein lysis buffer (1% Triton X-100, 150 mM NaCl, 10 mM Tris/HCl pH 7.4, 1 mM EDTA, 0.1% SDS containing 2 mM PMSF). Thereafter insoluble material was pelleted by centrifugation at 14000 g for 20 mins at 4° C. The clarified lysate was removed to a new tube for protein quantification and SDS-PAGE. The protein content in the supernatant fraction was determined using protein assay kits (Bio-Rad, Hemel Hempstead, UK). A total of 50 μg of protein was resuspended in 20 μl of sample buffer (125 mM Tris-HCl pH 6.8, 4% SDS, 5% 2-mercaptoethanol, 20% glycerol and 0.05% bromophenol blue) boiled for 5 mins at 95° C. and run on a 10% SDS-polyacrylamide gel prior to Western blotting.

Cells

Cells were seeded in 5 cm dishes and allowed to attach overnight. The following day, the cells were synchronised by incubating with serum-free medium for 24 hrs. Thereafter, the medium was replaced with fresh complete medium and the cells were grown in the presence or absence of DOX for 24, 48 and 72 hrs respectively. In parallel, cells were co-treated with indomethacin or NS-398. Cells were harvested by lysing in protein lysis buffer (150 mM NaCl, 10 mM Tris-HCl pH 7.4, 1 mM EDTA, 1% Triton X-100, 0.1% SDS). The protein content in the supernatant fraction was determined as described above. The clarified cell lysates (20 μg) were denatured and electrophoresed on 4-20% Tris-Glycine gels (NOVEX, Invitrogen).

Western Blotting

Immunoblot analysis was performed on supernatant fractions of cervical tissues and HeLa COX-1 Tet-Off cells. The proteins were transferred onto polyvinylidene difluoride membrane (PVDF, Millipore, Watford, UK) and subjected to immunoblot analysis. Membranes were blocked for 1 hr at 25° C. in 5% skimmed milk powder diluted in TBST (50 mM Tris-HCl, 150 mM NaCl and 0.05% v/v Tween-20). Thereafter, membranes were incubated overnight with either COX-1 (1:500), COX-2 (1:500), β-actin (1:500), PGES (1:250), bFGF (1:500), VEGF (1:500), Ang-1 (1:250) and Ang-2 (1:250) specific antibodies. Following transfer, membranes were subsequently incubated for 1 hr with rabbit anti-goat secondary antibody (for COX-1/2, β-actin, Ang-1/2, bFGF) at a dilution of 1:30000 or goat anti-rabbit secondary antibody (PGES, VEGF) at a dilution of 1:30000. Thereafter, membranes were washed in TBST and developed by the ECF chemiluminescence system following the manufacturers instructions. Proteins were revealed and quantified by PhosphorImager analysis using the STORM 860 system (Molecular Dynamics, UK). Fold induction in induced cells was determined relative to uninduced cells, after normalising to β-actin, by dividing the expression in induced cells by expression in uninduced cells. The molecular weights of the respective proteins were determined from the relative mobility on SDS-PAGE compared with molecular weight standards. COX-1 and COX-2 negative controls for determination of antibody specificity were performed by incubating membranes with goat anti-COX-1/2 antibody pre adsorbed to blocking peptide as per the manufacturers protocol. Data are presented as mean±SEM from 4 independent experiments.

Immunohistochemistry

The site of COX-1 expression was localised in cervical tissues by immunohistochemistry using archival cervical blocks (squamous cell carcinomas, C37-C47; adenocarcinomas, C48-C58 and normal cervix, N19-N23) obtained from the Department of Anatomical Pathology, University of Cape Town, South Africa. Five-micron paraffin wax-embedded tissue sections were cut and mounted onto coated slides (TESPA, Sigma). Sections were dewaxed in xylene, rehydrated in graded ethanol and washed in water followed by TBS (50 mM Tris-HCl, 150 mM NaCl pH 7.4) and blocked for endogenous endoperoxidase (1% H₂O₂ in methanol). Antigen retrieval was performed by pressure cooking for 2 mins in 0.01M sodium citrate pH 6. Sections were blocked using either 5% normal rabbit serum diluted in TBS. Subsequently the tissue sections were incubated with polyclonal goat anti-COX-1 antibody (sc-1752; Autogenbioclear) at a dilution of 1:200 at 4° C. for 18 hrs. Control tissue was incubated with goat anti-COX-1 antibody pre-adsorbed to blocking peptide (sc-1752p; Autogenbioclear) as per the manufacturer's protocol. After thorough washing with TBS, the tissue sections probed with the goat anti-human COX-1 primary antibody was incubated with biotinylated rabbit anti-goat secondary IgG antibody (Dako) at a dilution of 1:500 at 25° C. for 40 mins. Thereafter the tissue sections were incubated with streptavidin-peroxidase complex (Dako) at 25° C. for 20 mins. Colour reaction was developed by incubation with 3.3′-diaminobenzidine (Dako). The tissue sections were counterstained in aqueous hematoxylin, followed by sequential dehydration using graded ethanol and xylene, before mounting and coverslipping.

PGE₂ Assay

HeLa COX-1 Tet-Off cells were seeded in 5 cm dishes at a cell density of 5×10⁵ cells/dish and were allowed to grow and attach overnight. The following day, the cells were synchronised by incubating with serum-free medium for 24 hrs. COX-1 expression was induced for 24, 48 and 72 hrs respectively, by DOX withdrawal from the culture medium, in the presence or absence of indomethacin or NS-398. Arachidonic acid to a final concentration of 5 μg/ml was added to the culture medium following induction for 6 hrs. Thereafter, 1 ml of medium was removed and added to 1 ml of methyloximating solution. Control uninduced cells were treated similarly but maintained with DOX supplemented daily. PGE₂ secretion into the culture medium was assayed by ELISA (39). The ELISA was performed using 96 Well plates (Amine-binding plates; Costar, High Wycombe, UK) coated with donkey anti-rabbit antibody. Plates were then coated with rabbit immunoglobulin G (1 mg/ml diluted in PBS with 1% carbonate buffer, pH 9.6) at 200 μl/well for 16 hrs at 4° C. The solution was aspirated and blocking solution (50 mM glycine, 10 mg/ml bovine serum albumin) added at 25 μl/well for 2 hrs at 23° C. The plates were then washed and donkey anti-rabbit serum (Scottish Antibody Production Unit, Carluke, UK) added to a final volume of 150 μl/well, before washing, air drying and storage with desiccant at 4° C. The link was prepared by ether extraction and reverse phase chromatography using 20 mg of synthetic PGE₂, 320 μl dry dimethylformamide, 3 μl butylchloroformate and 0.05 mM biocytin. Samples and synthetic standards were diluted in ELISA buffer (150 mM NaCl, 100 mM Tris-HCl, 0.05% Tween-20, 50 mM phenol red, 1 mM 2-methylisothiazolone, 1 mM bromonitrodioxane, 2 mM EDTA, 2 mg/ml bovine serum albumin to a final pH 7.2) and 100 μl of each added in duplicate to the plate. The link was diluted 1:1.5×10⁶ in ELISA buffer and 50 μl added to each well. Antisera, diluted 1:50000 in ELISA buffer, was added to a final volume of 50 μl to all wells except those used for measuring non-specific binding. Plates were incubated at 4° C. for 16 his, washed and 100 μl/well of 0.2 unit/ml streptavidin-peroxidase was added. Plates were then incubated for 20 mins at 23° C. on an orbital shaker, washed and substrate (0.3 g/L urea-hydrogen peroxide, 0.1 g/L tetramethyl benzine in 10 mM sodium acetate, pH 6.0) added to a final volume of 200 μl/well for 10 mins before quenching with 50 μl/well 1M Sulfuric acid. Colour reaction was measured at 450 nm by spectrophotometry. The rabbit antiserum that was raised against PGE₂-complexed keyhole limpet hemocyanin has been previously characterised (40). Data are presented as mean±SEM from 3 independent experiments.

PGE₂ Stimulation and cAMP Measurement.

Functionality of the unregulated PGE₂ receptors was assessed by measuring cAMP accumulation following COX-1 induction in the presence or absence of indomethacin. Cells (2×10⁵) were plated in 6 well dishes containing 4 ml/well of complete medium containing DOX. Cells were allowed to attach overnight. The following day, the cells were synchronised by incubating with fresh medium containing no foetal bovine serum for 24 hrs. COX-1 Tet-Off cells were induced, by DOX withdrawal from the culture medium for 48 hrs at 37° C. in humidified 5% CO₂ in the presence or absence of indomethacin. In parallel, control uninduced cells were supplemented daily with DOX. Thereafter the culture medium was removed and replaced with serum-free medium containing IBMX (Sigma) to a final concentration of 1 mM for 40 mins at 37° C. Cells were then stimulated with 0 nM PGE₂ or 300 nM PGE₂ for 5, 10, 20 or 30 mins respectively. Following stimulation, the medium was removed and the cells lysed in 0.1M HCl. cAMP concentration was quantified by ELISA using a cAMP kit (Biomol, Affiniti, Exeter, UK) as per the manufacturer's protocol and normalised to protein concentration of the lysate. Protein concentrations were determined using protein assay kits (Bio-Rad). The data are presented as mean±SBM from 3 independent experiments.

Statistical Analysis

The data in this study were analysed by ANOVA using StatView 5.0 (Abacus Concepts, Berkeley, Calif.).

Results

Expression of COX-1 and COX-2 in Cervical Carcinomas and Normal Cervix.

Expression of COX-1 and COX-2 in cervical carcinomas was investigated using real-time quantitative RT-PCR (FIG. 1A) and Western blot analysis (FIG. 1B). Expression of COX-1 and COX-2 RNA were significantly up-regulated in 78% and 100% of cases of squamous cell carcinoma respectively and 100% of cases of adenocarcinoma investigated. By contrast, minimal COX-1 and COX-2 transcript were detected in normal cervical tissue by quantitative RT-PCR. COX-1 and COX-2 expression, as assessed by quantitative RT-PCR, was 19.9±5.9 and 118±32 fold greater in cervical carcinoma tissues than that observed in normal cervical tissue (P<0.01). Western blot analysis confirmed enhanced expression of COX-1 and COX-2 in cervical squamous cell carcinoma (85% and 100% of cases respectively; FIG. 1B, panel I) and adenocarcinoma (100% of cases respectively; FIG. 1B, panel II). Basal expression of COX-1 protein was detected in 87% of cases of normal cervix. No COX-2 expression was detected in normal cervical tissue by Western blot analysis (Fig. B, panel III). Specificity of detection of the 72 kDa COX-1 and COX-2 protein was performed by competition studies using a specific immunogen (blocking) peptide (data not shown).

Localisation of the Site of COX-1 Expression in Cervical Carcinomas and Normal Cervix.

The site of COX-1 expression in the carcinoma tissue was investigated by immunohistochemistry. COX-1 expression was up-regulated in all carcinoma samples. COX-1 was localised to the neoplastically transformed squamous epithelium in squamous cell carcinoma (FIG. 2A), and to neoplastically transformed columnar epithelium lining the endocervical canal and the glandular epithelium of the endocervical glands in adenocarcinomas (FIG. 2C). Little or no immunoreactivity for COX-1 was observed in the normal cervical tissues (FIG. 2E). Pre-adsorbing the antibody with the blocking peptide (COX-1 negative control) abolished the COX-1 immunoreactivity in all carcinoma samples. Representative sections incubated with the blocking peptide are shown in FIGS. 2B, 2D and 2F for squamous cell carcinoma, adenocarcinoma and normal cervical tissues respectively.

Inducible COX-1 Expression in HeLa Cells

To investigate the effect of COX-1 overexpression in HeLa neoplastic cervical epithelial cells, we established a DOX-regulated expression system. As shown in FIG. 3A, a 72 kDa immunoreactive COX-1 band was observed to increase in intensity 48 hrs following DOX withdrawal from the culture medium. Maximal sustained induction was achieved after 72 hrs. The fold induction for COX-1 overexpression above basal for 24, 48 and 72 hrs was determined to be 1.5±0.34, 3.7±0.45 and 4.7±0.56 fold respectively. COX-1 expression was normalised against β-actin on the same blot. Cells maintained in DOX for 72 hrs showed no elevation of COX-1 expression above basal. Pre-adsorbing the COX-1 antibody with the blocking peptide abolished the COX-1 immunoreactivity indicating specificity of the COX-1 antibody. These data indicate that high levels of inducible overexpression of COX-1 was achieved in HeLa cells. In order to determine whether COX-1 expression was altered by cell confluency or the addition of DOX to the culture medium, wild-type HeLa Tet-Off cells were grown for 72 hrs in the presence or absence of DOX. No increase in COX-1 expression above basal was observed suggesting that neither DOX nor cell density affected the expression of COX-1 in wild-type HeLa Tet-Off cells (FIG. 3A).

The functionality of the transfected COX-1 cDNA was assessed by measuring PGE₂ secretion into the culture medium following COX-1 induction for 24, 48 and 72 hrs respectively. A time-dependent increase in PGE₂ secretion into the culture medium accompanied the induction of COX-1 expression. PGE₂ production was significantly elevated after 48 hrs (272.2±18.8 nM; p<0.05) and 72 hrs (537±22.5 nM; p<0.01) when compared with PGE₂ levels in uninduced cells (118±6.75 nM; FIG. 3B). The addition of indomethacin reduced the PGE₂ levels to 62±7 nM and 76±10.7 nM after 48 hrs and 72 hrs respectively (p<0.01). Co-treatment of cells with NS-398 (selective COX-2 inhibitor) partially reduced PGE₂ levels to 132±26.2 and 268±17 nM after 48 hrs and 72 hrs respectively (p<0.05).

COX-1 Overexpression Induces COX-2 and PGES

COX-enzyme products including PGE₂ are known to induce COX-2 expression (22). In order to investigate the effect of COX-1-enzyme products on expression of COX-2 and the microsomal glutathione-dependent inducible PGES, COX-1 Tet-Off HeLa cells were grown in the presence or absence of the dual COX-enzyme inhibitor indomethacin or the highly selective COX-2 inhibitor NS-398 for 24, 48 and 72 hr. Following DOX withdrawal from the culture medium, a time-dependent increase in COX-1 overexpression was observed with maximal sustained overexpression after 72 hrs (FIG. 4A). Concomitant with this increase in COX-1 expression was a 3.2±8.9 fold increase in COX-2 expression after 72 hrs and a 2.5±0.45 and 1.3±0.78 fold increase in PGES after 24 hrs and 48 hrs respectively (FIG. 4A). After 72 hrs PGES levels had returned to basal. Co-treatment of the HeLa cells, induced for 24, 48 and 72 hrs respectively, with indomethacin or NS-398 showed no alteration in COX-1 overexpression (FIGS. 4B and 4C). However indomethacin treatment inhibited COX-2 as well as PGES induction (FIG. 4B). No significant change in COX-2 expression was observed after treatment of HeLa cells with NS-398 (FIG. 4C). Induction of PGES by COX-1 overexpression was delayed by 24 hrs following treatment of HeLa cells with NS-398 (FIG. 4C).

COX-1 Overexpression in HeLa Cells Induces PGE₂ Receptor Expression

The effect of COX-1 over-expression on the four subtypes of PGE₂ receptors, namely EP1-EP4, was investigated by real-time quantitative RT-PCR, following DOX withdrawal from the culture medium and subsequent induction of COX-1. Induced overexpression of COX-1 for 24, 48 and 72 hrs had no significant effect on EP1 receptor expression when compared with cells grown in the presence of indomethacin. COX-1 overexpression for 48 and 72 hrs significantly induced expression of EP2 receptor transcript when compared with indomethacin treated cells (FIG. 5, p<0.01). Levels of EP3 receptor transcript were significantly induced after 48 hrs of COX-1 overexpression (p<0.05) compared with cells grown in the presence of indomethacin. EP4 receptor transcript was significantly up-regulated after COX-1 overexpression for 24, 48 and 72 hrs compared with cells co-treated with the COX-enzyme inhibitor (p<0.05).

cAMP Production in COX-1 Overexpressing Cells in Response to PGE₂

The effect of COX-1 induced up-regulation of the cAMP-linked PGE₂ receptors on cAMP production was determined following overexpression of COX-1 and stimulation with exogenous PGE₂. No significant difference in basal cAMP production was detected in uninduced and induced cells (FIG. 6). Treatment of uninduced cells with 300 nM PGE₂ resulted in a 2.43±1.07 fold increase in cAMP production (p<0.05). Cells in which COX-1 was induced for 48 hrs prior to stimulation with exogenous PGE₂ showed a rapid transient 12.67±3.7 fold cAMP response (p<0.01). This rapid elevated cAMP production in COX-1 overexpressing cells in response to PGE₂ was abolished when cells were grown in medium containing the COX-enzyme inhibitor indomethacin. The activity of EP1 receptor was investigated by measuring inositol phosphate accumulation (41). No inositol phosphate accumulation above basal was observed in COX-1 Tet-Off HeLa cells following induced expression of COX-1 and PGE₂ stimulation (data not shown).

Induction of Angiogenic Factors in Response to COX-1 Overexpression

The effect of COX-1 on expression of the angiogenic factors bFGF, VEGF, Ang-1 and Ang-2 was assessed by Western blot analysis. Overexpression of COX-1 for 72 hrs resulted in a 2.3±0.45 fold increase in bFGF (FIG. 7A), a 4.5±1.2 fold increase in VEGF (FIG. 7B), a 2.3±0.78 fold increase in Ang-1 (FIG. 7C) and a 2.1±0.98 fold increase in Ang-2 expression (FIG. 7D) respectively. Indomethacin treatment inhibited the COX-1 associated up-regulation of bFGF, VEGF, Ang-1 and Ang-2 (FIG. 7). Treatment of cells with NS-398 partially reduced the up-regulation of bFGF, VEGF, Ang-1 and Ang-2 expression (FIG. 7) suggesting that products from both COX enzymes were modulating expression of these factors.

Discussion

Recent studies have demonstrated up-regulated and inducible expression of COX-1 in different biological models. COX-1 expression is up-regulated in human breast cancer (24), human prostate cancer (25) and murine models of lung tumourigenesis (23). In addition, COX-1 expression can be induced in vitro by tobacco carcinogen (42), VEGF (21), arachidonic acid, forskolin, dibutryl-cAMP and PGE₂ (22). In an in vitro model, COX-1 overexpression in endothelial cells implanted in mice was associated with enhanced tumourigenicity (5). This study demonstrates up-regulation of COX-1 expression in squamous cell carcinoma and adenocarcinoma of the human cervix as demonstrated by real-time quantitative RT-PCR, Western blot analysis and immunohistochemistry. The up-regulation of expression of COX-1 was associated with enhanced expression of COX-2. Moreover, the site of COX-1 expression localised to the neoplastic epithelial cells of all squamous cell carcinomas and adenocarcinomas investigated, demonstrating a similar pattern of expression for COX-1 in cancer of the cervix as has been demonstrated for COX-2 (16, 18) and PGE₂ (18). These data suggest that both COX-enzymes and/or their products may contribute towards the development of cervical cell neoplasias.

In order to investigate the effect of overexpression of COX-1, we have established a DOX-regulated expression system in HeLa cells. Initial studies performed on wild-type HeLa Tet-Off cells showed no elevation of COX-1 expression above basal when wild-type cells were grown for 72 hrs in the presence or absence of DOX. These data demonstrate that neither cell growth nor DOX affected the basal expression of COX-1. Overexpression of COX-1 in HeLa cells up-regulates expression of COX-2 and PGES concomitant with increased PGE₂ production. These data suggest that COX-2 and inducible PGES are co-regulated. In an in vitro model system, administration of interleukin (IL)-1β to A549 cells rapidly induced the expression of COX-2 and PGES (43). Similarly, inducible PGES activity has been described in lipopolysaccharide-stimulated rat peritoneal macrophages, coincident with COX-2 expression and PGE₂ biosynthesis (44, 45). Indomethacin, but not NS-398, treatment abolished the up-regulation of expression of COX-2 and PGES and synthesis of PGE₂. Up-regulation of COX-2 and PGES in HeLa cells may thus be mediated by prostanoids produced following overexpression of COX-1. NS-398 treatment significantly reduced PGE₂ synthesis at 72 hrs but not 48 hrs. This is not surprising, since COX-2 expression in HeLa cells was only maximally induced at 72 hrs. This suggests that PGE₂ production detected at 72 hrs after COX-1 overexpression is enhanced by the activity of both COX enzymes. In other model systems, COX-2 expression is up-regulated by PGE₂ via the cAMP-dependent PGE₂-receptors (22). In vitro studies have shown that cAMP activity accompanies a concomitant increase in COX-2 synthesis suggesting that cAMP is the primary secondary messenger in regulating COX-2, presumably via the upstream cAMP response element (CRE) located on the COX-2 gene (46). The biological actions of PGE₂ have been attributed to its interaction with G-protein-coupled receptors, of which 4 subtypes (EP1-4) have been identified (35). COX-1 overexpression in HeLa cells resulted in significant up-regulation of the cAMP-dependent PGE₂-receptors after 48 hrs of COX-1 overexpression. This up-regulation was inhibited by growing cells in medium containing indomethacin, suggesting that the up-regulation was mediated by COX-enzyme products. Previous studies have demonstrated enhanced PGE₂ synthesis in cervical carcinomas together with up-regulated expression of EP2 and EP4 receptors and enhanced cAMP-responsiveness of cervical tumour tissue to PGE₂ (18). Since COX-1 overexpression in HeLa cells induces COX-2 and EP receptor expression, it is feasible that PGE₂, may facilitate the process of cervical tumourigenesis in an autocrine/paracrine manner following enhanced EP receptor expression and ligand-receptor interaction. A direct role for EP receptors in tumourigenesis has been reported recently in colon cancer cells. In this model, enhanced proliferative and tumourigenic effects were mediated by PGE₂ following interaction with the EP4 receptor (47). It is likely that similar mechanisms may exist in cervical carcinomas to enhance growth and proliferation via EP receptors in a cAMP-dependent manner. Since both COX enzymes catalyse the same reaction, enzyme-products such as PGE₂ from both COX enzymes may regulate EP receptor expression. The choice of COX enzyme for biosynthesis of prostaglandins may depend on the relative expression of each COX isoform in the cell, as in many cells, COX-2 levels are typically only 20%-30% of COX-1 (46).

Functionality of the induced EP receptors in our model system was assessed by measuring cAMP in response to stimulation with exogenous PGE₂. cAMP activity was measured in HeLa cells following overexpression of COX-1 for 48 hrs and stimulation with exogenous PGE₂, A significant fold increase in cAMP production was observed after 5 min of PGE₂ stimulation in COX-1 induced compared with uninduced cells. This augmented cAMP response was abolished by growing cells in medium containing indomethacin. These data suggest that PGE₂ produced by COX-1 overexpression may be acting in an autocrine/paracrine manner via the cAMP-linked PGE₂-receptors to mediate its effect on target genes, such as COX-2, via the PKA pathway by activating adenylate cyclase and increasing cAMP. Since COX-1 overexpression had no significant effect on EP1 expression and stimulation of HeLa cells with PGE₂ resulted in no increase in inositol phosphate accumulation above basal, this suggested that although PGE₂ may be functioning via EP 1 receptors coupled to inositol phosphate production and release of intracellular calcium in these cells, its contribution to events associated with COX-1 up-regulation was minimal.

Cancer cells produce a wide variety of factors that contribute to angiogenesis, including bFGF, VEGF, bFGF-binding protein and platelet-derived growth factor (PDGF) (34). Our data demonstrate that COX-1 overexpression in HeLa cells results in the up-regulation of expression of pro-angiogenic factors. Induced overexpression of COX-1 resulted in an increase in bFGF, VEGF, Ang-1 and Ang-2 expression. Co-treatment of these cells with indomethacin abolished the up-regulation of these angiogenic factors. This suggests that the up-regulation of these factors is mediated by prostanoids produced by COX-1 overexpression. Moreover, since the effects of COX-1 overexpression can be reversed by COX inhibition with indomethacin, this confirms that these affects are not an artefact of forced overproduction of the enzyme. Partial reduction in expression of these factors by treatment with NS398 suggests that both enzymes (COX-1 and COX-2) converge to regulate expression of target genes possibly through common prostanoid synthetic pathways. In another model system, COX-2 overexpression and increase in PGE₂ synthesis in colon carcinoma cells results in the up-regulation of bFGF and VEGF and this is associated with arrangement of endothelial cells into tubular structures (34). The up-regulation of angiogenic factors by COX enzymes is important in regulating angiogenesis and maintenance of the neoplastic tissue. As the demand for nutrients and oxygen increases for tissue development, an increased vascularisation is necessary to supply nutrients to the tumour (48). In this study, we also observe the regulation of the angiogenic factors Ang-1 and Ang-2 by COX enzymes. Ang-1 is a Tie-2 receptor agonist, which is required for recruitment of perivascular cells leading to the formation and stabilisation of capillaries, vessel maturation and endothelial cell survival (49, 50). Ang-1 and other angiogenic factors such as VEGF may act synergistically to increase vascular sprouting and branching (51, 52). In addition Ang-1/Tie-2 interaction enhances the mitogenic effect of VEGF on endothelial cell growth (53). By contrast, Ang-2 is a natural Tie-2 receptor antagonist, destabilising cell contacts and thus allowing access to angiogenic factors such as VFGF (54). In our model system, enhanced synthesis of prostanoids as a consequence of up-regulated COX-1 may thus act in an autocrine/paracrine manner to up-regulate the expression of COX-2 and target receptors as well as the intracellular signalling to a host of angiogenic factors, which could act on endothelial cells and lead to the recruitment of new blood vessels to enhance tumour mass.

TABLE 1
Extent of invasiveness of cervical carcinoma biopsy
samples of South African women.

| Sample no. | Histological typing | FIGO stage |
|---|---|---|
| C10 C14; C28-C32 | Squamous | 1B; well differentiated |
| | carcinoma | |
| C5-C9, C24-C27, | Squamous | 2B; well differentiated |
| C37-C47 | carcinoma | |
| C1-C4; C19-C23 | Squamous | 3B; well differentiated |
| | carcinoma | |
| C36 | Adenocarcinoma | 1B; moderately differentiated |
| C15-C18, C33- | Adenocarcinoma | 2B; well differentiated |
| C35, | | |
| C48-C58 | | |

### EXAMPLE 2

### Cyclooxygenase-2 Expression and Prostaglandin E2 Synthesis are Up-Regulated in Carcinomas of the Cervix: a Possible Autocrine/Paracrine Regulation of Neoplastic Cell Function via EP2/EP4 Receptors

The purpose of this study was to determine whether cyclooxygenase-2 (COX-2) expression and prostaglandin E2 (PGE₂) synthesis are up-regulated in cervical cancers. Real-time quantitative RT-PCR and Western blot analysis confirmed COX-2 RNA and protein expression in all cases of squamous cell carcinoma (n=8) and adenocarcinoma (n=2) investigated. In contrast minimal expression of COX-2 was detected in histologically normal cervix (n=5). Immunohistochemical analyses localised COX-2 expression and PGE₂ synthesis to neoplastic epithelial cells of all squamous cell (n=1) and adenocarcinomas (n=1O) studied. Immunoreactive COX-2 and PGE₂ were also co-localised to endothelial cells lining the microvasculature. Minimal COX-2 and PGE₂ immunoreactivity were detected in normal cervix (n=5). In order to establish whether PGE₂ has an autocrine/paracrine effect in cervical carcinomas, we investigated the expression of two subtypes of PGE₂ receptors, namely EP2 and EP4, by real-time quantitative RT-PCR. Expression of EP2 and EP4 receptors was detected in cervical squamous cell carcinoma (n=7) and adenocarcinoma (n=1) and was significantly higher than that detected in histologically normal cervix (n=5; P<0.01). Finally, the functionality of the EP2/EP4 receptors was assessed by investigating cAMP generation following in vitro culture of cervical cancer biopsies and normal cervix in the presence or absence of 300 nM PGE₂. cAMP production was detected in all carcinoma tissue following treatment with exogenous PGE₂ and was significantly higher in carcinoma tissue (n=7) than that detected in normal cervix (n=5; P<0.05). The fold induction of cAMP in response to PGE₂ was determined to be 51.1±12.3 in cervical carcinoma tissue compared with 5.8±2.74 in normal cervix. These results confirm that COX-2, EP2, EP4 expression and PGE₂ synthesis are up-regulated in cervical cancer tissue and suggest that PGE₂ may regulate neoplastic cell function in cervical carcinoma in an autocrine/paracrine manner via the EP2/EP4 receptors.

Introduction

Cancer of the uterine cervix is one of the leading causes of cancer-related death in women world-wide. It is reported as being particularly common in less developed countries, including South and Central America, Southeast Asia and Sub-Saharan Africa (1, 2, 3), where 80% of the world's cervical cancers occur (4). Three histological categories of epithelial tumours of the cervix are recognised by the World Health Organisation (5). These are squamous-cell carcinoma, adenocarcinoma and other less common-types of epithelial tumours. The most common histological type of cervical carcinoma is squamous cell carcinoma that accounts for 60%-80% of all cervical cancers. Adenocarcinoma accounts for approximately 20% of invasive cervical carcinoma.

Cyclooxygenase (COX) enzymes, also called prostaglandin endoperoxide synthase, catalyse the rate limiting step in the conversion of arachidonic acid to prostaglandin H₂ and other eicosanoids including prostaglandin E (6). There are at least two isoforms of the COX enzyme, COX-1 and COX-2 (7, 8). COX-1 is constitutively expressed in many tissues and cell types and generates prostaglandins for normal physiological function (8). By contrast, the expression of COX-2 is rapidly induced following stimulation of quiescent cells by growth factors, oncogenes, carcinogens and tumour-promoting phorbal esters (7, 8, 9). Prostaglandin E2 (PGE₂) elicits its autocrine/paracrine effects on target cells through interaction with seven transmembrane G-protein-coupled-receptors (GPCR) which belong to the rhodopsin family of serpentine receptors (10). Four main sub-types of PGE₂ receptors have been identified (EP₁, EP₂, EP₃, EP₄) which utilise alternate and in some cases opposing intracellular pathways (11). To-date, the role of the different PGE₂ receptors, their divergent intracellular signalling pathways as well as their target genes involved in mediating the effects of PGE₂ on normal or neoplastically transformed cervical epithelium remain to be elucidated.

Recently, a relationship between COX-2, its synthesised product PGE₂, and neoplastic transformation of epithelial cells has been established (12, 13). Transcription of COX-2 is upregulated in numerous cancers including colon, pancreas, oesophagus, lung, prostate and bladder (14, 15, 16, 17, 18, 19). It has been proposed that COX-2 overexpression and PGE₂ synthesis mediate neoplastic transformation of epithelial cells by increasing their proliferation rate, resistance to apoptosis and invasiveness. These effects are mediated by suppressing the transcription of target genes tat may be involved in cellular growth/transformation (eg. p53) and adhesion (eg. E-Cadherin) (13, 20). Moreover, COX-2 and PGE₂ promote cancer development and invasiveness by mediating the transcription of angiogenic factors that promote both migration of endothelial cells and their arrangements into tubular structures (21, 22).

The present study was designed to investigate whether COX-2 expression and PGE₂ synthesis are upregulated in human squamous cell carcinomas and adenocarcinomas of the cervix. In addition, a possible autocrine/paracrine role for PGE₂ in cervical carcinogenesis was assessed by investigating (a) the expression of EP2/EP4 receptors in cervical carcinoma tissue and (b) the effect of exogenous treatment of carcinoma tissue with PGE₂ on cAMP turnover.

Materials and Methods

Tissue Collection and Processing

Cervical specimens were obtained at the time of surgery/biopsy from patients that were attending the Gynaecologic Oncology Clinic at Groote Schuur Hospital, Cape Town and that had previously been diagnosed with invasive carcinoma of the cervix. Punch biopsies were taken from the lesion by an experienced Gynaecologist with a special interest in oncology. A portion of the biopsy was excised and fixed in formalin followed by paraffin wax-embedding for histopathological typing. The remaining portion was snap frozen in either dry ice or liquid nitrogen and stored at −70° C. for RT-PCR and Western blot analysis or transported at 4° C. for in vitro culture and PGE₂ stimulation. Histologically normal cervical samples were obtained from patients undergoing surgery for non-malignant conditions. The ages of the patients ranged from 29 years to 81 years with a median age of 50.5 years. The study was approved by the University of Cape Town Research Ethics Committee and informed consent was obtained from all patients before tissue collection. The data in this study were analysed by ANOVA using StatView 5.0.

Real-Time Quantitative PCR

Real-time quantitative RT-PCR was performed to assess COX-2, EP2 and EP4 expression. RNA samples were extracted from cervical tissue (n=7-8 squamous cell carcinoma, n=1-2 adenocarcinoma and n=5 normal cervix) using TA-Reagent (Sigma Chemical Company, Dorset, UK) as per the manufacturers protocol. RNA, samples were reverse transcribed using MgCl₂ (5.5 mM), dNTPs (0.5 mM each), random hexamers (1.25 μM), oligo-dT (1.25 μM), RNAase inhibitor (0.4 U/μl) and multiscribe reverse transcriptase (1.2 U/μl; all from PE Biosystems, Warrington, UK). The mix was aliquoted into individual tubes (16 μl/tube) and template RNA was added (4 l/tube of 100 ng/μl RNA). Samples were incubated for 60 minutes at 25° C., 45 minutes at 48° C. and then at 95° C. for 5 minutes. A reaction mix was made containing Taqman buffer (5.5 mM MgCl₂, 200 μM dATP, 200 μM dCTP, 200 μM dGTP, 400 μM dUTP), ribosomal 18S forward and reverse primers and probe (all at 50 nM), forward and reverse primers for COX-2, EP2 or EP4 receptor (300 nM), COX-2, EP2 or EP4 receptor probe (200 nM), AmpErase UNG (0.01 U/μl) and AmpliTaq Gold DNA Polymerase (0.025 U/μl; all from PE Biosystems). A volume of 48 μl of reaction mix was aliquoted into separate tubes for each cDNA sample and 2 μl/replicate of cDNA was added. After mixing 23 μl of sample were added to the wells on a PCR plate. Each sample was added in duplicate. A no template control (containing water) was included in triplicate. Wells were sealed with optical caps and the PCR reaction run on an ABI Prism 7700 using standard conditions. COX-2 and EP receptor primers and probe for quantitative PCR were designed using the PRIMER express program (PE Biosystems). The sequence of the COX-2 primers and probe were as follows: Forward: 5′-CCT TCC TCC TGT GCC TGA TG-3′ (SEQ ID No 13); Reverse: 5′-ACA ATC TCA TTT GAA TCA GGA AGC T-3′ (SEQ ID No 14); Probe (FAM labelled): 5′-TGC CCG ACT CCC TTG GGT GTC A-3′ (SEQ ID No 15). The sequence of the EP2 receptor primers and probe were as follows; Forward: 5′-GAC CGC TTA CCT GCA GCT OTA C-3′ (SEQ ID No 19); Reverse: 5′-TGA AGT TGC AGG CGA GCA-3′ (SEQ ID No 20); Probe (FAM labelled): 5′-CCA CCC TGC TGC TGC TTC TCA TTG TCT-3′ (SEQ ID No 21). The sequence of the EP4 receptor primers and probe were as follows; Forward: 5′-ACG CCC CCT ACT CCT ACA TG-3′ (SEQ ID No 25); Reverse: 5′-AGA GGA CGG TGG CGA GAA T-3′ (SEQ ID No 26); Probe (FAM labelled): 5′-ACG CGG GCT TCA GCT CCT TCC T-3′ (SEQ ID No 27). The ribosomal 18S primers and probe sequences were as follows; Forward: 5′-CGC CTA CCA CAT CCA AGG AA-3′ (SEQ ID No 28); Reverse: 5′-GCT GGA ATT ACC GCG GCT-3′ (SEQ ID No 29); Probe (VIC labelled): 5′-TGC TGG CAC CAG ACT TGC CCT C-3′ (SEQ ID No 30). Expression of COX-2, EP2 and EP4 was normalised to RNA loading for each sample using the 18s ribosomal RNA as an internal standard. Relative gene expression in carcinoma tissue compared with normal cervix was calculated by dividing the expression in carcinoma tissue by expression in the normal cervix. The data are presented as mean±SEM.

Western Blotting

COX-2 protein expression was assessed by Western blotting. Proteins were extracted from cervical tissue (n=8 squamous cell carcinoma; n=2 adenocarcinoma and n=5 normal cervix) using Tri-Reagent (Sigma) following the manufacturers instructions. A total of 100 μg of protein was resuspended in 38 μl of sample buffer (125 mM Tris-HCl pH 6.8, 4% SDS, 5% 2-mercaptoethanol, 20% glycerol and 0.05% bromophenol blue) boiled for 5 minutes at 95° C. and run on a 10% SDS-polyacrylamide gel. Proteins were transferred onto polyvinylidene difluoride membrane (PVDF, Millipore, Watford, UK) and subjected to immunoblot analysis. Membranes were blocked for 1 hour at 25° C. in 5% skimmed milk powder diluted in washing buffer (50 mM Tris-HCl, 150 mM NaCl and 0.05% v/v Tween-20). Thereafter, membranes were incubated with goat anti-COX-2 primary IgG antibody (sc-1745; Autogenbioclear, Wiltshire, UK) at a dilution of 1:500 at 4° C. for 18 hours. Control samples were incubated with goat anti-COX-2 antibody pre-adsorbed to blocking peptide (sc-1745p; Autogenbioclear) according to the manufacturer's protocol. Membranes were subsequently incubated for 1 hour respectively with rabbit anti-goat secondary IgG antibody conjugated to biotin (Dako, High Wycombe, UK) (1:500) and streptavidin-biotin horseradish peroxidase complex (Amersham, Aylesbury, UK). Proteins were revealed by chemiluminescence (ECLplus kit; Amersham) following the manufacturers instructions. The molecular weight of the COX-2 protein was determined as being approximately 72 kDa from the relative mobility on SDS-PAGE compared with the molecular weight standard.

Immunohistochemistry

The site of COX-2 expression and PGE₂ synthesis was localised in cervical tissues by immunohistochemistry using archival cervical blocks (n=10 squamous cell carcinoma; n=10 adenocarcinoma and n=5 normal cervix) obtained from the Department of Anatomical Pathology, University of Cape Town, South Africa. Five-micron paraffin wax-embedded tissue sections were cut and mounted onto coated slides (TESPA, Sigma). Sections were dewaxed in xylene, rehydrated in graded ethanol and washed in water followed by TBS (50 mM Tris-HCl, 150 mM-NaCl pH 4) and blocked for endogenous endoperoxidase (1% H₂O₂ in methanol). Antigen retrieval was performed by pressure cooking for 2 minutes in 0.1 M sodium citrate pH 6 (for COX-2 and PGE₂). No antigen retrieval was performed for CD34 immunohistochemistry. Sections were blocked using either 5% normal rabbit serum (for COX-2), 5% swine serum (for PGE₂) or 5% normal goat serum (for CD34) diluted in TBS. Subsequently the tissue sections were incubated with polyclonal goat anti-COX-2 antibody (sc-1745; Autogenbioclear) at a dilution of 1:400, rabbit anti-PGE₂ antibody (kindly supplied by Professor R W Kelly, MRC Human Reproductive Sciences Unit, Edinburgh, UK) at a dilution of 1:100 or monoclonal mouse anti-human CD34 primary antibody (mca-547; Serotec, Oxford, UK) at a dilution of 1:25 at 4° C. for 18 hours. Control tissue was incubated with either 5% antisera (PGE₂ and CD34) or goat anti-COX-2 antibody pre-adsorbed to blocking peptide (sc-1745p; Autogenbioclear). After thorough washing with TBS, the tissue sections probed with the goat anti-human COX-2 and rabbit anti-PGE₂ primary antibodies were incubated with biotinylated rabbit anti-goat secondary IgG antibody (for COX-2; Dako) or swine anti-rabbit secondary IgG antibody (for PGE₂; Dako) at a dilution of 1:500 at 25° C. for 40 minutes. Thereafter the tissue sections were incubated with streptavadin-biotin peroxidase complex (Dako) at 25° C. for 20 minutes. Tissue sections probed with the mouse anti-human CD34 antibody were developed using a Mouse EnVision Kit (Dako) as per the manufacturers instructions, Colour reaction was developed by incubation with 3.3′-diaminobenzidine (Dako). The tissue sections were counterstained in aqueous hematoxylin, followed by sequential dehydration using graded ethanol and xylene, before mounting and coverslipping.

PGE₂ Stimulation and CAMP Measurement.

Determination of Basal CAMP levels in cervical tissues. Initially, basal cAMP levels were measured in cervical tissue (n=7 squamous cell carcinomas and n=5 normal cervix; FIG. 12A). Carcinoma and normal cervical tissues were obtained on the day of surgery/biopsy, sectioned finely and divided equally into three aliquots. The tissue was transported at 4° C. and then incubated in 35 mm tissue culture dishes containing 2 ml of Dulbecco's Modified Eagle Medium (DMEM) (Sigma), containing 10% foetal calf serum, 0.3 mg/ml L-glutamine, 100 IU penicillin and 100 μg streptomycin for 1.5 hours. One aliquot of tissue was snap frozen to determine basal cAMP concentration in the tissue at the time of collection. The other two aliquots were incubated overnight at 37° C. in humidified 5% CO₂ in the presence or absence of 3 μg/ml indomethacin [a dual COX enzyme inhibitor (24, 32)]. Subsequently, tissue sections were harvested by centrifugation at 2000 g. The supernatant was discarded and the tissue homogenised in 0.1 M HCl. cAMP concentration was quantified by ELISA using a cAMP kit (Biomol; Affiniti, Exeter, UK) as per the manufacturer's protocol and normalised to protein concentration of the homogenate, Protein concentrations were determined using protein assay kits (Bio-Rad, Hemel Hempstead, UK).

cAMP Production in Cervical Tissues in Response to Exogenous PGE₂:

Cervical tissues (n=6 squamous carcinoma, n=1 adenocarcinoma and n=5 normal cervix) were sectioned finely, divided equally into three aliquots and incubated overnight in Dulbecco's Modified Eagle Medium (DMEM) (Sigma), containing 10% foetal calf serum, 0.3 mg/ml L-glutamine, 100 IU penicillin and 100 μg streptomycin and 3 μg/ml indomethacin. Following overnight incubation samples were incubated in the same medium containing IBMX (Sigma) to a final concentration of 1 mM for 30 minutes at 37° C. and then stimulated with 0 nM PGE_{2, 300} nM PGE₂ or 50 μM forskolin (forskolin treatment in sample C2 was excluded due to the small size of the biopsy) for 5 minutes. Tissue sections were harvested-by centrifugation at 2000 g. The supernatant was discarded and the tissue homogenised in 0.1 M HCl. cAMP concentration and protein concentrations were determined as mentioned above

Results

Expression of COX-2 in cervical carcinomas was investigated using real-time quantitative RT-PCR (FIG. 8A) and Western blot analysis (FIG. 5B). Expression of COX-2 was signifcantly up-regulated in all cases of squamous cell carcinoma and adenocarcinoma investigated. COX-2 expression as assessed by quantitative RT-PCR was 150.8±43.18 greater in cervical carcinoma tissues than that observed in normal cervical tissue (P<0.05). Western blot analysis on these cervical carcinomas revealed immunoreactive bands of approximately 72 kDa. Minimal levels of COX-2 transcipt was detected in normal cervical tissue by quantitative RT-PCR and no COX-2 protein was detected in any of the normal cervical samples. Preadsorbing the primary antibody with the blocking peptide abolished the COX-2 signal in the carcinoma samples thus confirming the specificity of the detection of the 72 kDa COX-2 protein in the carcinoma samples.

The site of COX-2 expression and PGE₂ synthesis in the carcinoma tissue was investigated by immunohistochemistry. Immunoreactive COX-2 and PGE₂ were upregulated in all carcinoma samples. COX-2 and PGE₂ were localised to the neoplastically transformed squamous epithelium in squamous cell carcinoma (FIGS. 9a and 9b respectively), and to neoplastically transformed columnar epithelium lining the endocervical canal and the glandular epithelium of the endocervical glands in adenocarcinomas (FIGS. 9c and 9d respectively). In addition, COX-2 and PGE₂ immunostaining were observed in endothelial cells lining the vasculature in all squamous cell carcinoma and adenocarcinoma sections investigated (FIGS. 10a and 10b). To confirm that COX-2 expression and PGE₂ synthesis were localised to the endothelial cells of blood vessels, immunohistochemistry was performed on tissue sections using antibodies raised against the CD34 endothelial cell marker. The pattern of expression with CD34 was identical to that observed with COX-2 and PGE₂ thus confirming that COX-2 expression and PGE₂ synthesis is localised to the endothelial cell layer of blood vessels in human cervical carcinoma (FIG. 3c). Negligible staining was observed in the stromal compartment in all carcinoma tissue investigated. Moreover, little or no staining for COX-2 and PGE₂ was observed in the normal cervical tissues (FIGS. 9e and 9f respectively). Preadsorbing the antibody with the blocking peptide (COX-2 negative control) or incubating sections with non-immune serum (PGE₂ negative control) abolished the COX-2 and PGE₂ signal in all carcinoma samples (representative samples are shown for COX-2 and PGE₂ in FIGS. 9g and 9h respectively). No CD34 staining was observed in sections incubated with non-immune serum in place of primary antibody (FIG. 10d).

The expression of two subtypes of PGE₂ receptors, namely EP2 and EP4, was investigated by real-time quantitative RT-PCR in cervical carcinoma and normal cervix (FIG. 11). Expression of both receptors was significantly up-regulated in all carcinoma tissues compared with normal cervix (P<0.01). The relative expression of EP2 and EP4 receptor in carcinoma tissue was 14.5±3.2 and 106±25.8 (respectively) greater than that detected in normal cervix. In order to assess the activity of the EP2/EP4 receptors in the cervical tissue, basal levels of cAMP were determined at the time of tissue collection and after overnight incubation in the absence or presence of 3 μindomethacin (FIG. 12A). cAMP concentration immediately after tissue excision was significantly higher in carcinoma compared with normal cervix (77.9±30.9 vs 32.5±8.7 pmol cAMP/mg protein; P<0.05). cAMP concentrations in carcinoma tissue following overnight incubation in the absence of indomethacin was similar to that detected in the tissue at the time of excision (64.2±5.1 pmol cAMP/mg protein) but was significantly reduced when the tissue was cultured in the presence of indomethacin (2.59±0.64 pmol cAMP/mg protein). In normal cervical tissue, levels of cAMP were significantly reduced following overnight incubation in the absence or presence of indomethacin (11.96±1.35 and 4.0±0.7 pmol cAMP/mg protein respectively; P<0.05). Subsequently, we determined the effect of exogenous PGE₂ and forskolin treatment on cAMP production in carcinoma and normal cervical tissues (FIG. 12B). Stimulation of cervical carcinoma tissue with 300 nM PGE₂ or 50 μM forskolin (positive control) yielded a greater cAMP response than in normal cervical tissue treated in the same manner. Overall, the fold induction of CAMP generation after PGE₂ and forskolin stimulation was 51.1±12.3 and 55.3±15.84 respectively in cancer tissue and 5.8±1.68 and 9.18±1.59 respectively in normal cervix (p<0.01).

Discussion

This study demonstrates upregulation of COX-2 and PGE₂ production in squamous cell carcinoma and adenocarcinoma of the human cervix as demonstrated by real-tie quantitative RT-PCR, Western blot analysis and immunohistochemistry. These data suggest a similar pattern of expression of COX-2 in cancer of the cervix as has been demonstrated in other carcinomas (14, 15, 16, 17, 18, 19). In addition, in this study increased COX-2 expression is associated with increased synthesis of PGE₂ as both COX-2 and PGE₂ co-localised in neoplastic epithelial cells and endothelial cells of the microvasculature. Previous studies have suggested that PGE₂ is the predominant prostaglandin synthesised from arachidonic acid by COX-2 (25). Enhanced synthesis of PGE₂ resulting from upregulated COX-2 could induce malignant change in epithelial cells through immunosuppression (6), inhibiting apoptosis (20), increasing metastatic potential of epithelial cells (26) and promoting angiogenesis (21, 22). COX-2 and PGE₂ control the process of angiogenesis in tumours either directly or indirectly. In an in vitro model, overexpression of COX-2 and PGE₂ in colon epithelial cells enhances the expression of angiogenic factors that act on endothelial cells resulting in enhanced cell migration and microvascular tube formation (21). More recently, it was suggested that COX-2-and PGE₂ produced by endothelial cells may also directly regulate the process of angiogenesis (22). The arrangement of rat aortic endothelial cells into tubular structures is reduced following treatment with selective COX-2 inhibitors and this effect is partially reversed by co-treatment with PGE₂ (22). Hence, it is feasible to suggest that in cervical carcinomas the process of angiogenesis is regulated by COX-2 and PGE₂ through an epithelial-endothelial and/or endothelial-endothelial cell interaction. This is supported by our data demonstrating COX-2 expression and PGE₂ synthesis in neoplastic epithelial cells as well as endothelial cells.

PGE₂ acts on target cells through interaction with G-protein coupled receptors. To-date several of these receptors have been cloned which utilise alternate intracellular signalling pathways. In this study we investigated the expression of two PGE₂ membrane-bound receptors, namely EP2 and EP4, which mediate their effect on target cells via the PKA pathway by activating adenylate cyclase and increasing intracellular cAMP (10). In cervical carcinomas, expression of EP2 and EP4 receptors is up-regulated compared with normal cervix. In addition, the basal cAMP concentration in the carcinoma tissue is elevated compared with normal cervix.-Treatment of the cervical tissue with the COX enzyme inhibitor indomethacin significantly reduced cAMP concentration. This suggests that the elevated basal cAMP concentration in the carcinoma tissue is mediated by COX enzyme products. Moreover, treatment of cervical carcinoma tissue with exogenous PGE₂ or forskolin following overnight incubation with the COX-enzyme inhibitor indomethacin, results in a rapid cAMP response which is greater in the carcinoma tissue than in the normal cervical tissue. This suggests up-regulation of adenylate cyclase in cervical carcinomas compared with normal cervix and that prostaglandins via EP2/EP4 receptors may be involved in cervical neoplasia. The exact role of COX-2 and relative contributions of prostanoids, including PGE₂ in cervical neoplasia has yet to be established. However it is reasonable to suggest that PGE₂ secreted by neoplastic epithelial and/or endothelial cells may act on EP2/EP4 receptors in cervical carcinoma in an autocrine/paracrine manner to enhance tumourigenesis.

COX-2 inhibitors exhibit dramatic antineoplastic activity in a number of tumour model systems investigated thus far, including: colon cancer cells implanted into nude mice, tumour production in APC mutant mice and carcinogen-induced tumours in rats (27, 28, 29). This is mediated partially, by reducing PGE₂ synthesis in the COX-2 over-expressing cells which in turn down-regulates the survival, metastatic and angiogenic potential of the cancerous tissue (20, 21, 26). This has prompted the suggestion that the inhibition of PGE₂ secretion by the application of COX-2 inhibitors may have an effect on growth and invasiveness of various carcinomas (21, 22, 26, 27). Such treatments may also be of benefit in regulating the growth of cervical carcinoma. Treatment of cervical carcinoma with NSAIDs will suppress endogenous expression of COX-2 and synthesis of PGE₂ which may act in an autocrine/paracrine manner via the EP2/EP4 receptors. However, it is important to emphasise that in sexually active women the use of selective COX-2 inhibitors may be of partial therapeutic benefit. In these women growth and invasiveness of neoplastic cells may be also under the direct influence of PGE₂ present in seminal plasma. Prostaglandin concentration in seminal plasma is 10000 times higher than that found at the site of inflammation and PGE is the predominant type of prostaglandin detected (30). Future studies to elucidate the relative contributions of endogenous and seminal plasma prostaglandins on the phenotypic behaviour of neoplastically transformed cervical epithelial and endothelial cells may assist in implementing improved therapy for women with cervical carcinomas.

### EXAMPLE 3

### Up-Regulation of Expression of Cyclooxygenase-2 and Prostaglandin E Receptors (EP2 and EP4) in HeLa Cells by Seminal Plasma

We have demonstrated up-regulated expression of COX-2 and enhanced synthesis of PGE{sub}2{/sub} in cervical carcinomas (see Example 2). Enhanced PGE₂ synthesis as a consequence of COX-2 overexpression has been associated with various carcinomas and is regarded as a promoter of neoplastic cell proliferation and angiogenesis. In sexually active women growth and invasiveness of neoplastic cervical cells may be also under the direct influence of PGE present in seminal plasma. The aim of this study was to investigate the effect of seminal plasma on the expression of COX-2 and the PGE₂ receptor subtypes EP2 and EP4 in HeLa (cervical epithelial) cells. The expression of COX-2, EP2 and EP4, was measured by real-time RT-PCR. Treatment of HeLa cells with seminal plasma for 24 hrs resulted in up-regulation of expression of COX-2, EP2 and EP4 receptors (fold induction above basal was 20.2+9.26, 12.5±6.2 and 7.8±2.8 respectively, p less than 0.05). Co-treatment-of cells with the dual COX-enzyme inhibitor indomethacin abolished the upregulation in EP2/EP4 receptor expression. Treatment of cells with the selective COX-2 inhibitor NS-398, and the PKA inhibitor H-89 partially abolished the up-regulated receptor expression. Subsequently, we investigated the effect of seminal plasma on cAMP signalling in HeLa cells, as COX-2 expression is regulated via the cAMP-pathway, presumably via the cAMP-response element on the COX-2 promotor (Smith et al (2000) Annu. Rev. Biochem. 69,145-182). Stimulation of HeLa cells for 5 min with seminal plasma yielded a 12.8±5.68 fold increase in cAMP production compared with unstimulated cells (P less than 0.05). These data raise the possibility that, in sexually active women, seminal plasma may act in an autocrine/paracrine manner to modulate the COX-2/PGE₂ biosynthetic pathway and could thus play a role in regulating neoplastic cell function.

### EXAMPLE 4

### Treating Cervical Cancer with an Inhibitor of COX-1

A patient, diagnosed with invasive cervical carcinoma is administered ibuprofen or SC560 or mofezolac.

### EXAMPLE 5

### Treating a Neoplastic Condition of the Cervix with an EP4 Receptor Antagonist

A patient diagnosed with CIN III is administered 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one.

### EXAMPLE 6

### Treating Cervical Cancer with a Combination of COX-1 Inhibitor and EP2 Receptor Antagonist

A patient diagnosed with cervical carcinoma in situ is administered the combination of AH23848B and sulindac or SC560 or mofezolac.

### EXAMPLE 7

### Suppository

| | mg/suppository |
|---|---|
| AH22921 (63 μm)* | 250 |
| Hard Fat, BP (Witepsol H15 - Dynamit Nobel) | 1770 |
| | 2020 |

*The antagonist AH22921 is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved Maintaining the mixture at 45° C., the remaining Witepsol H15 is added to the suspension and stirred to ensure a homogenous mix. The entire suspension is passed through a 250 μM stainless steel screen and, with continuous siring, is allowed to cool to 40° C. At a temperature of 38° C. to 40° C. 2.02 g of the mixture is filled into suitable plastic moulds. The suppositories are allowed to cool to room temperature.

### EXAMPLE 8

### Pessaries

| | mg/pessary |
|---|---|
| AH23848B | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

### EXAMPLE 9

### Vaginal Ring

A vaginal ring containing 5-butyl-2.4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt; is produced using core extrusion technology.

### EXAMPLE 10

### Intrauterine Device

An intrauterine device containing AH6809 is produced using standard technology. The device is for use with women diagnosed with CIN I, II or III.

### EXAMPLE 11

### Tampon

A tampon for halting the progression from CIN II to CIN III is produced by impregnating a standard tampon with an effective dose of 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

The EP2 or EP4 receptor antagonist of Examples 7 to 11 may be used in combination with a COX-1 inhibitor, or may be replaced with another EP2 or EP4 receptor anatgonist or with a COX-1 inhibitor.

### REFERENCES FOR EXAMPLE 1

1. Sitas, F., Madhoo, J., and Wessie, J. Incidence of histologically diagnosed cancer in South Africa, 1993-1995. Johannesburg: National Cancer Registry of South Africa, South African Institute for Medical Research., 1998.

2. Scully, R. E., Bonfiglio, T. A., Kurman, R. J., Silverberg, S. G., and Wilkinson, E. J. Histological typing of female genital tract tumors., p. 189. Berlin: Springer-Verlag, 1994.

3. DuBois, R. N., Radhika, A., Reddy, B. S., and Entingh, A. J. Increased cyclooxygenase-2 levels in carcinogen-induced rat colonic tumors. Gastroenterology, 110: 1259-1262, 1996.

4. Eberhart, C. E., Coffey, R. J., Radhika, A., Giardiello, F. M., Ferrenbach, S., and DuBois, R. N. Up-regulation of cyclooxygenase 2 gene expression in human colorectal adenomas and adenocarcinomas. Gastroenterology, 107: 1183-1188, 1994.

5. Narko, K., Ristimaki, A., MacPhee, M., Smith, E., Haudenschild, C. C., and Hla, T. Tumorigenic transformation of immortalized ECV endothelial cells by cyclooxygenase-1 overexpression. J Biol Chem, 272: 21455-21460, 1997.

6. Tsujii, M., Kawano, S., and DuBois, R. N. Cyclooxygenase-2 expression in human colon cancer cells increases metastatic potential. Proc Natl Acad Sci USA, 94: 3336-3340, 1997.

7. Williams, C. S., Tsujii, M., Reese, J., Dey, S. K., and DuBois, R. N. Host cyclooxygenase-2 modulates carcinoma growth. J Clin Invest, 105: 1589-1594, 2000.

8. Hla, T. and Neilson, K. Human cyclooxygenase-2 cDNA. Proc Natl Acad Sci USA, 89: 7384-7388, 1992.

9. Herschman, H. R. Regulation of prostaglandin synthase-1 and prostaglandin synthase-2. Cancer Metastasis Rev, 13: 241-256, 1994.

10. Vane, J. R., Bakhle, Y. S., and Botting, R. M. Cyclooxygenases 1 and 2. Annu Rev Pharmacol Toxicol, 38: 97-120, 1998.

11. Kargman, S. L., O'Neill, G. P., Vickers, P. J., Evans, J. F., Mancini, J. A., and Jothy, S. Expression of prostaglandin G/H synthase-1 and -2 protein in human colon cancer. Cancer Res, 55: 2556-2559, 1995.

12. Herschman, H. R. Prostaglandin synthase 2. Biochim Biophys Acta, 1299: 125-140, 1996.

13. Tsujii, M. and DuBois, R. N. Alterations in cellular adhesion and apoptosis in epithelial cells overexpressing prostaglandin endoperoxide synthase 2, Cell, 83: 493-501, 1995.

14. Tucker, 0. N., Dannenberg, A. J., Yang, E. K., Zhang, F., Teng, L., Daly, J. M., Soslow, R. A., Masferrer, J. L., Woerner, B. M., Koki, A. T., and Fahey, T. J., 3rd Cyclooxygenase-2 expression is up-regulated in human pancreatic cancer. Cancer Res, 59: 987-990, 1999.

15. Wolff, H., Saukkonen, K., Anttila, S., Karjalainen, A., Vainio, H., and Ristimaki, A. Expression of cyclooxygenase-2 in human lung carcinoma. Cancer Res, 58: 4997-5001, 1998.

16. Kulkarni, S., Rader, J. S., Zhang, F., Liapis, H., Koki, A. T., Masferrer, J. L., Subbaramaiah, K., and Dannenberg, A. J. Cyclooxygenase-2 is overexpressed in human cervical cancer Clin Cancer Res, 7: 429-434., 2001.

17. Ryu, H. S., Chang, K. H., Yang, H. W., Kim, M. S., Kwon, H. C., and Oh, K. S. High cyclooxygenase-2 expression in stage IB cervical cancer with lymph node metastasis or parametrial invasion. Gynecol Oncol, 76: 320-325, 2000.

18. Sales, K. J., Katz, A. A., Davis, M., Hinz, S., Soeters, R. P., Hofmeyr, M. D., Millar, R. P., and Jabbour, H. N. Cyclooxygenase-2 expression and prostaglandin E(2) synthesis are up-regulated in carcinomas of the cervix: a possible autocrine/paracrine regulation of neoplastic cell function via EP2/EP4 receptors. J Clin Endocrinol Metab, 86: 2243-2249., 2001.

19. Brannon, T. S., North, A. J., Wells, L. B., arid Shaul, P. W. Prostacyclin synthesis in ovine pulmonary artery is developmentally regulated by changes in cyclooxygenase-1 gene expression. J Clin Invest, 93: 2230-2235, 1994.

20. Smith, C. J., Morrow, J. D., Roberts, L. J. d., and Marnett, L. S. Differentiation of monocytoid THP-1 cells with phorbol ester induces expression of prostaglandin endoperoxide synthase-1 (COX-1). Biochem Biophys Res Commun, 192: 787-793, 1993.

21. Bryant, C. E., Appleton, I., and Mitchell, J. A. Vascular endothelial growth factor upregulates constitutive cyclooxygenase 1 in primary bovine and human endothelial cells. Life Sci, 62: 2195-2201, 1998.

22. Maldve, R. E., Kim, Y., Muga, S. J., and Fischer, S, M. Prostaglandin E(2) regulation of cyclooxygenase expression in keratinocytes is mediated via cyclic nucleotide-linked prostaglandin receptors. J Lipid Res, 41: 873-881, 2000.

23. Bauer, A. K., Dwyer-Nield, L. D., and Malkinson, A. M. High cyclooxygenase 1 (COX-1) and cyclooxygenase 2 (COX-2) contents in mouse lung tumors. Carcinogenesis, 21; 543-550, 2000.

24. Hwang, D., Scollard, D., Byrne, J., and Levine, E. Expression of cyclooxygenase-1 and cyclooxygenase-2 in human breast cancer. J Natl Cancer Inst, 90: 455-460, 1998.

25. Kirschenbaum, A., Klausner, A. P., Lee, R., Unger, P., Yao, S., Liu, X., and Levine, A. C. Expression of cyclooxygenase-1 and cyclooxygenase-2 in the human prostate. Urology, 56: 671-676, 2000.

26. Marnett, L. J. Aspirin and the potential role of prostaglandins in colon cancer. Cancer Res, 52: 5575-5589, 1992.

27. Murakami, M., Naraba, H., Tanioka, T., Semmyo, N., Nakatani, Y., Kojima, F., Ikeda, T., Fueki, M., Ueno, A., Oh, S., and Kudo, I. Regulation of prostaglandin E2 biosynthesis by inducible membrane associated prostaglandin E2 synthase that acts in concert with cyclooxygenase-2. J Biol Chem, 275: 32783-32792., 2000.

28. Forsberg, L., Leeb, L., Thoren, S., Morgenstern, R., and Jakobsson, P. Human glutathione dependent prostaglandin E synthase: gene structure and regulation. FEBS Lett, 471: 78-82, 2000.

29. Simonson, M. S., Herman, W. H., and Dunn, M. J. PGE2 induces c-fos expression by a cAMP-independent mechanism in glomerular mesangial cells. Exp Cell Res, 215: 137-144, 1994.

30. Baylink, T. M., Mohan, S., Fitzsimmons, R. J., and Baylink, D. J. Evaluation of signal transduction mechanisms for the mitogenic effects of prostaglandin E2 in normal human bone cells in vitro. J Bone Miner Res, 11: 1413-1418, 1996.

31. Yano, T., Yano, Y., Uchida, M., Murakami, A., Hagiwara, K., Otani, S., and Ichikawa, T. The modulation effect of vitamin E on prostaglandin E2 level and ornithine decarboxylase activity at the promotion phase of lung tumorigenesis in mice. Biochem Pharmacol, 53: 1757-1759, 1997.

32. DeWitt, D. L. Prostaglandin endoperoxide synthase: regulation of enzyme expression. Biochim Biophys Acta, 1083: 121-134, 1991.

33. Jones, M. K., Wang, H., Peskar, B. M., Levin, E., Itani, R. M., Sarfeh, I. J., and Tarnawski, A. S. Inhibition of angiogenesis by nonsteroidal anti-inflammatory drugs: insight into mechanisms and implications for cancer growth and ulcer healing. Nat Med, 5: 1418-1423, 1999.

34. Tsujii, M., Kawano, S., Tsuji, S., Sawaoka, H., Hori, M., and DuBois, R. N. Cyclooxygenase regulates angiogenesis induced by colon cancer cells. Cell, 93: 705-716, 1998.

35. Coleman, R. A., Smith, W. L., and Narumiya, S. International Union of Pharmacology classification of prostanoid receptors: properties, distribution, and structure of the receptors and their subtypes. Pharmacol Rev, 46: 205-229, 1994.

36. Ashby, B. Co-expression of prostaglandin receptors with opposite effects: a model for homeostatic control of autocrine and paracrine signaling. Biochem Pharmacol, 55: 239-246, 1998.

37. FIGO T. N. M. Atlas, 3rd edition, 2nd revision edition, p. 196. Heidelberg: Springer-Verlag, 1992.

38. Gossen, M. and Bujard, H. Tight control of gene expression in mammalian cells by tetracycline-responsive promoters. Proc Natl Acad Sci USA, 89: 5547-5551, 1992.

39. Denison, F. C., Calder, A. A., and Kelly, R. W. The action of prostaglandin E2 on the human cervix: stimulation of interleukin 8 and inhibition of secretor leukocyte protease inhibitor. Am J Obstet Gynecol, 180; 614-620, 1999.

40. Kelly, R. W., Graham, B. J., and O'Sullivan, M. J. Measurement of PGE2 as the methyl oxime by radioimmunoassay using a novel iodinated label. Prostaglandins Leukot Essent Fatty Acids, 37: 187-191, 1989.

41. Berg, K. A., Clarke, W. P., Sailstad, C., Saltzman, A., and Maayani, S. Signal transduction differences between 5-hydroxytryptamine type 2A and type 2C receptor systems. Mol Pharmacol, 46: 477-484, 1994.

42. Rioux, N. and Castonguay, A. The induction of cyclooxygenase-1 by a tobacco carcinogen in U937 human macrophages is correlated to the activation of NF-kappaB. Carcinogenesis, 21: 1745-1751, 2000.

43. Jakobsson, P. J., Thoren, S., Morgenstern, R., and Samuelsson, B. Identification of human prostaglandin E synthase: a microsomal, glutathione-dependent, inducible enzyme, constituting a potential novel drug target. Proc Natl Acad Sci USA, 96: 7220-7225, 1999.

44, Naraba, H., Murakami, M., Matsumoto, H., Shimbara, S., Ueno, A., Kudo, I-, and Oh-ishi, S. Segregated coupling of phospholipases A2, cyclooxygenases, and terminal prostanoid synthases in different phases of prostanoid biosynthesis in rat peritoneal macrophages J Immunol, 160: 2974-2982, 1998.

45. Matsumoto, H., Naraba, H., Murakami, M., Kudo, I., Yamaki, K., Ueno, A., and Oh-ishi, S. Concordant induction of prostaglandin E2 synthase with cyclooxygenase-2 leads to preferred production of prostaglandin E2 over thromboxane and prostaglandin D2 in lipopolysaccharide-stimulated rat peritoneal macrophages. Biochem Biophys Res Commun, 230: 110-114, 1997.

46. Smith, W. L., DeWitt, D. L., and Garavito, R. M. CYCLOOXYGENASES, structural, cellular, and molecular biology. Annu Rev Biochem, 69: 145-182, 2000.

47. Sheng, H., Shao, J., Washington, M. K, and DuBois, R. N. Prostaglandin E2 increases growth and motility of colorectal carcinoma cells. J Biol Chem, 12: 12, 2001.

48. Seed, M. P., Brown, J. R., Freemantle, C. N., Papworth, J. L., Colville-Nash, P. R., Willis, D., Somerville, K. W., Asculai, S., and Willoughby, D. A. The inhibition of colon-26 adenocarcinoma development and angiogenesis by topical diclofenac in 2.5% hyaluronan. Cancer Res, 57: 1625-1629, 1997.

49. Papapetropoulos, A., Garcia-Cardena, G., Dengler, T. J., Maisonpierre, P. C., Yancopoulos, G. D., and Sessa, W. C. Direct actions of angiopoietin-1 on human endothelium: evidence for network stabilization, cell survival, and interaction with other angiogenic growth factors. Lab Invest, 79: 213-223, 1999.

50. Asahara, T., Chen, D., Takahashi, T., Fujikawa, K., Kearney, M., Magner, M., Yancoponlos, G. D., and Isner, J. M. Tie2 receptor ligands, angiopoietin-1 and angiopoietin-2, modulate VEGF-induced postnatal neovascularization [see comments]. Circ Res, 83: 233-240, 1998.

51. Carmeliet, P., Ferreira, V., Breier, G., Pollefeyt, S., Kieckens, L., Gertsenstein, M., Fahrig, M., Vandenhoeck, A., Harpal, K., Eberhardt, C., Declercq, C., Pawling, J., Moons, L., Collen, D., Risau, W., and Nagy, A. Abnormal blood vessel development and lethality in embryos lacking a single VEGF allele. Nature, 380: 435-439, 1996.

52. Koblizek, T. I., Weiss, C., Yancopoulos, G. D., Deutsch, U., and Risau, W. Angiopoietin-1 induces sprouting angiogenesis in vitro. Curr Biol, 8: 529-532, 1998.

53. Huang, X. L., Takakura, N., and Suda, T. In vitro effects of angiopoietins and VEGF on hematopoietic and endothelial cells, Biochem Biophys Res Commun, 264: 133-138, 1999.

54. Maisonpierre, P. C., Suri, C., Jones, P. F., Bartunkova, S., Wiegand, S. J., Radziejewski, C., Compton, D., McClain, J., Aldrich, T. H., Papadopoulos, N., Daly, T. J., Davis, S., Sato, T. N., and Yancopoulos, G. D. Angiopoietin-2, a natural antagonist for Tie2 that disrupts in vivo angiogenesis. Science, 277: 55-60, 1997.

### REFERENCES FOR EXAMPLE 2

1. Munoz N, Bosch F X, de Sanjose S, et al . 1992 The causal link between human papillomavirus and invasive cervical cancer: a population-based case-control study in Colombia and Spain. Int J Cancer. 52:743-749.

2. Aareleid T, Pukkala E, Thomson H, Hakama M. 1993 Cervical cancer incidence and mortality trends in Finland and Estonia; a screened vs. an unscreened population. Eur J Cancer. 5:745-749.

3. Sitas F, Carrara H I, Terblanche M, Madhoo J. 1997 Screening for cancer of the cervix in South Africa. S Afr Med J. 87:620-622.

4. Beral V, Hermon C, Munoz N, Devesa S S. 1994 Cervical cancer. vol. 19. Cold Spring Harbour Press.

5. Scully R E, Bonfiglio T A, Kurman R J, Silverberg S G, Wilkinson E J. 1994 Histological typing of female genital tract tumors. Springer-Verlag, Berlin.

6. DeWitt D L. 1991 Prostaglandin endoperoxide synthase: regulation of enzyme expression. Biochim Biophys Acta. 1083:121-134.

7. Hla T, Neilson K 1992 Human cyclooxygenase-2 cDNA. Proc Natl Acad Sci USA. 89:7384-7388.

8. Herschman H R. 1996 Prostaglandin synthase 2. Biochim Biophys Acta. 1299:125-140.

9. Subbaramaiah K, Telang N, Ramonetti J T, et al. 1996 Transcription of cyclooxygenase-2 is enhanced in transformed mammary epithelial cells. Cancer Res. 56:4424-4429.

10. Coleman R A, Smith W L, Narumiya S. 1994 International Union of Pharmacology classification of prostanoid receptors. properties, distribution, and structure of the receptors and their subtypes. Pharmacol Rev. 46:205-229.

11. Ashby B. 1998 Co-expression of prostaglandin receptors with opposite effects: a model for homeostatic control of autocrine and paracrine signaling. Biochem Pharmacol. 55:239-246.

12. Sheng H, Shao J, Morrow J D, Beauchamp R D, DuBois R N. 1998 Modulation of apoptosis and Bcl-2 expression by prostaglandin E2 in human colon cancer cells. Cancer Res. 58:362-366.

13. Subbaramaiah K, Altorki N, Chung W J, Mestre J R, Sampat A, Dannenberg A J. 1999 Inhibition of cyclooxygenase-2 gene expression by p53. J Biol Chem. 274:10911-10915.

14. Zimmermann K C, Sarbia M, Weber A A, Borchard F, Gabbert B E, Schror K 1999 Cyclooxygenase-2 expression in human esophageal carcinoma, Cancer Res. 59:198-204.

15. Tucker O N, Dannenberg A J, Yang E K, et al. 1999 Cyclooxygenase-2 expression is upregulated in human pancreatic cancer. Cancer Res. 59:987-990.

16. Ratnasinghe D, Tangrea J, Roth M J, et al. 1999 Expression of cyclooxygenase-2 in human squamous cell carcinoma of the esophagus; an immunohistochemical survey. Anticancer Res. 19:171-174.

17. Wolff H, Saukkonen K, Anttila S, Karjalainen A, Vainio H, Ristimaki A. 1998 Expression of cyclooxygenase-2 in human lung carcinoma. Cancer Res. 58:4997-5001.

18. Gupta S, Srivastava M, Ahmad N, Bostwick D G, Mukhtar H. 2000 Over-expression of cyclooxygenase-2 in human prostate adenocarcinoma. Prostate. 42:73-78.

19. Mohammed S I, Knapp D W, Bostwick D G, et al. 1999 Expression of cyclooxygenase-2 (COX-2) in human invasive transitional cell carcinoma (TCC) of the urinary bladder. Cancer Res. 59:5647-5650.

20. Tsujii M, DuBois R N. 1995 Alterations in cellular adhesion and apoptosis in epithelial cells overexpressing prostaglandin endoperoxide synthase 2. Cell. 83:493-501.

21. Tsujii M, Kawano S, Tsuji S, Sawaoka H, Hori M, DuBois RN. 1998 Cyclooxygenase regulates angiogenesis induced by colon cancer cells. Cell. 93:705-716.

22. Jones M K, Wang B, Peskar B M, et al. 1999 Inhibition of angiogenesis by nonsteroidal anti-inflammatory drugs: insight into mechanisms and implications for cancer growth and ulcer healing. Nat Med, 5:1418-1423.

23. Marnett L J. 1992 Aspirin and the potential role of prostaglandins in colon cancer. Cancer Res. 52:5575-5589.

24. Masferrer J L, Zweifel B S, Manning P T, et al. 1994 Selective inhibition of inducible cyclooxygenase 2 in vivo is antinflammatory and nonulcerogenic. Proc Natl Acad Sci USA. 91:3228-3232.

25. Brock T G, McNish R W, Peters-Golden M. 1999 Arachidonic acid is preferentially metabolized by cyclooxygenase-2 to prostacyclin and prostaglandin E2. J Biol Chem. 274:11660-11666.

26. Tsuji M, Kawano S, DuBois R N. 1997 Cyclooxygenase-2 expression in human colon cancer cells increases metastatic potential. Proc Natl Acad Sci USA. 94:3336-3340.

27. Oshima M, Dinchuk J E, Kargman S L, et al. 1996 Suppression of intestinal polyposis in Apc delta716 knockout mice by inhibition of cyclooxygenase 2 (COX-2). Cell. 87:803-809.

28. Sheng H, Shao J, Kirkland S C, et al. 1997 Inhibition of human colon cancer cell growth by selective inhibition of cyclooxygenase-2. J Clin Invest. 99:2254-2259.

29. Kawamori T, Rao CV, Seibert K, Reddy B S. 1998 Chemopreventive activity of celecoxib, a specific cyclooxygenase-2 inhibitor, against colon carcinogenesis. Cancer Res. 58:409412.

30. Templeton A A, Cooper I, Kelly R W. 1978 Prostaglandin concentrations in the semen of fertile men. J Reprod Fertil. 52:147-150.

1. A method of treating a neoplastic condition of the cervix in a patient the method comprising administering to the patient an inhibitor of cyclooxygenase-1 (COX-1).

2. A method of treating a neoplastic condition of the cervix in a patient the method comprising administering to the patient an EP2 or EP4 receptor antagonist.

3. A method of treating a neoplastic condition of the cervix in a patient the method comprising administering to the patient an inhibitor of cyclooxygenase-1 (COX-1) and an EP2 or EP4 receptor antagonist.

4. A method according to claims 1 and 3 wherein the inhibitor of COX-1 is any one or more of the NSAIDs ibuprofen, naproxen, fenbufen, fenoprofen, flurbiprofen, ketoprofen, dexketoprofen, tiaprofenic acid, azapropazone, diclofenac, aceclofenac, diflunisal, etodolac, indomethacin, ketorolac, mefenamic acid, meloxicam, namubetone, phenylbutazone, piroxicam, sulindac, tenoxicam, tolfenamic acid, SC560 and mofezolac.

5. A method according to claims 2 or 3 wherein the EP2 or EP4 receptor antagonist is any one or more of AH6809, an omega-substituted prostaglandin E derivative described in WO 00/15608 (Ono Pharm Co Ltd), AH23848B, AH22921X, IFTSYLECL (SEQ ID No 1), IFASYECL (SEQ ID No 2), IFTSAECL (SEQ ID No 3), IFTSYEAL (SEQ ID No 4), ILASYECL (SEQ ID No 5), IFTSTDCL (SEQ ID No 6), TSYEAL (SEQ ID No 7) (with 4-biphenylalanine), TSYEAL (SEQ ID No 8) (with homophenylalanine), a 5-thia-prostaglandin E derivative described in WO 00/03980 (Ono Pharm Co Ltd), 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl)-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, and 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

6. A method according to any one of the preceding claims wherein the neoplastic condition of the cervix is any one of CIN grade I, CIN grade II, CIN grade III, carcinoma in situ and invasive carcinoma.

7. Use of an inhibitor of COX-1 in the manufacture of a medicament for treating a neoplastic condition of the cervix in a patient.

8. Use of an EP2 or EP4 receptor antagonist in the manufacture of a medicament for treating a neoplastic condition of the cervix in a patient.

9. Use according to claim 7 wherein the patient is administered an EP2 and/or EP4 receptor antagonist.

10. Use according to claim 8 wherein the patient is administered an inhibitor of COX-1.

11. Use of any one or more of (a) an inhibitor of COX-1 and (b) an EP2 or EP4 receptor antagonist for treating a neoplastic condition of the cervix.

12. Any one or more of (a) an inhibitor of COX-1 and (b) an EP2 or EP4 receptor antagonist for use in treating a neoplastic condition of the cervix.

13. A therapeutic system for treating a neoplastic, condition of the cervix, the system comprising a combination of any two or more of (a) an inhibitor of COX-1 and (b) an EP2 or EP4 receptor antagonist.

14. A therapeutic system for treating a neoplastic condition of the cervix, the system comprising any one or more of (a) an inhibitor of COX-1 and (b) an EP2 or EP4 receptor antagonist adapted for delivery to the cervix.

15. A vaginal ring or a tampon or an intrauterine device comprising an EP2 and/or an EP4 receptor antagonist and/or an inhibitor of COX-1.

16. A combination of any one or more of an EP2 and/or EP4 receptor antagonist and an inhibitor of COX-1 to treat a neoplastic condition of the cervix.

17. A pharmaceutical composition comprising a combination according to claim 16 and a pharmaceutically acceptable carrier.

18. Any novel method of treating neoplastic condition of the cervix as herein described.
